(19) United States
(12) Patent Application Publication
(10) Pub. No.: US 2025/0009694 A1
(43) Pub. Date: Jan. 9, 2025

(54) METHODS AND PHARMACEUTICAL COMPOSITIONS FOR REPAIRING INTESTINAL MUCOSAL

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITÉ D'AIX MARSEILLE, Marseille (FR)

(72) Inventors: Philippe NAQUET, Marseille Cedex 09 (FR); Franck GALLAND, Marseille Cedex 09 (FR); Virginie MILLET, Marseille Cedex 09 (FR)

(21) Appl. No.: 18/702,021

(22) PCT Filed: Oct. 31, 2022
(86) PCT No.: PCT/EP2022/080332

§ 371 Date: Apr. 17, 2024

(30) Foreign Application Priority Data
Nov. 2, 2021 (EP) 21306537.8

(51) Int. Cl. A61K 31/197; A61K 31/145; A61K 31/16; A61P 1/00

(52) U.S. Cl. CPC A61K 31/197; A61K 31/145; A61K 31/16; A61P 1/00

(57) ABSTRACT

In the management of patients with inflammatory bowel diseases (IBD), there is a need to identify druggable biological pathways to improve mucosal repair and efficacy of TNF alpha biologics. Based on the VIVA transgenic model of Vnn1 overexpression on intestinal cells, the inventors show that the epithelial pantetheinase Vnn1 has a dual effect on colon: (1) its enzymatic products, cysteamine and pantothenic acid (vitamin B5) enhance coenzyme A regeneration and colon fitness through metabolic rewiring: (2) they favor microbiota-dependent accumulation of butyrate, previously shown to regulate mucosal energetics and to be reduced in IBD patients. Upon dextran sodium sulfate (DSS)-induced colitis, Vnn1 exerts a cytoprotective role on colonocytes and reinforces the mucosal barrier. Remarkably, this global pro-healing phenotype is recapitulated by treating control mice with the substrate (pantethine) or the products of pantetheinase activity prior to exposure to DSS. Therefore, enhancement of vitamin B5-driven metabolism should improve mucosal healing and maintain colon fitness and might enhance the efficacy of anti-inflammatory anti-TNF alpha therapy.

### FIELD OF THE INVENTION

The present invention relates to pharmaceutical fied. More particularly, the invention relates to use of an activator of vitamin B5 metabolism for improving mucosal repair.

### BACKGROUND OF THE INVENTION

Inflammatory bowel diseases (IBD) mostly encompass two disabling, lifelong and incurable disorders, Crohn's disease (CD) and ulcerative colitis (UC) [1]. Epidemiological studies indicate that their incidence and prevalence are constantly growing in developed countries [2]. Currently, anti-TNF alpha biologics represent the primary treatment of IBD [3, 4]. However, this therapy fails in 30% patients due to a non-responsiveness or the progressive development of a resistance to therapy after an initial response, thus imposing the search for new strategies [5-8]. The persistence of a mucosal inflammation correlates with the relapses and complications of IBD, and could be due to an incomplete histological remission and healing [9, 10]. The structural basis of mucosal healing is the restitution of an intact barrier function of the gut epithelium that prevents the translocation of commensal bacteria towards the submucosa with subsequent immune cell activation [11, 12]. Disruption of the intestinal barrier and repeated epithelial damage, both hallmarks of IBD, become new targets in the management of this chronic disease [13, 14].

An intact mucosal barrier requires the adequate control of epithelial cell fitness, cell-junctions and specific functions such as the luminal secretion of mucus or anti-microbial peptides [15, 16]. Transcriptomic and proteomic analyses of gut mucosa in IBD revealed metabolic alterations in colonocytes, including a shift from oxidative phosphorylation to glycolysis and a deficit in lipid-dependent energy production [17-20]. These changes might depend on the impaired production of short-chain fatty acids (SCFAs) by the gut microbiota [21, 22]. Indeed, butyrate is required for the regulation of stem cell renewal and represents the major energetic substrate through the coenzyme A (CoA)-dependent process of fatty acid β-oxidation (FAO) in mature colonocytes [23, 24]. This suggests that colonocytes might be particularly sensitive to variations in CoA levels.

In eukaryotes, CoA cannot diffuse across membranes and must by synthesized in each cell from cysteine, ATP and pantothenate (vitamin B5) [25]. Eukaryotic cells are unable to synthesize pantothenate and uptake it via the SMVT transporter from extracellular supply. Consequently, pantothenate must derive from the degradation of the (acyl-) CoA present in the food or the gut microbiome prior to its intestinal absorption, or from the systemic recycling of cell-derived CoA [25]. In each case, its availability depends on the efficacy of extracellular CoA degradation, a process in which the Vnn pantetheinases play a non-redundant function by hydrolysing CoA-derived pantetheine (PanSH) into cysteamine (CEA) and pantothenate (Pan) [25-27].

Our pioneering studies in mouse showed that the Vnn1 pantetheinase isoform regulates metabolic, inflammatory and cytoprotective programs in diverse tissues [28-32]. In gut, Vnn1 is abundantly expressed in ileum but present at low levels on colonocytes where its expression is inducible by various stresses [33-35]. Likewise, the colonic epithelium of IBD patients displays high VNN1 levels [34]. In addition, SNPs in the regulatory regions of the VNN1 gene are associated with disease risk and some VNN1 high patients harbouring specific VNN1 SNPs develop a severe disease [34].

In the management of patients with inflammatory bowel diseases (IBD), there is a need to identify prognostic markers and druggable biological pathways to improve mucosal repair and efficacy of TNF alpha biologics.

Herein the inventors show that enhancement of vitamin B5-driven metabolism should improve mucosal healing and maintain colon fitness and might enhance the efficacy of anti-inflammatory anti-TNF alpha therapy. They also found significantly augmented levels of indoxyl sulfate (IS) in urine of IBD patients compared to control patients.

### SUMMARY OF THE INVENTION

In a first aspect, the present invention relates to methods and pharmaceutical compositions for improving intestinal mucosal barrier repair in subject in need thereof. The present invention relates to methods and pharmaceutical compositions for treatment of gut inflammatory diseases such as inflammatory bowel diseases.

In a second aspect, the present invention relates to methods for diagnose inflammatory bowel diseases.

In particular, the present invention is defined by the claims.

### DETAILED DESCRIPTION OF THE INVENTION

To investigate the role of Vnn1 in susceptibility to colitis, the inventors extended their initial observation by monitoring VNN1 expression in a new cohort of IBD patients stratified according to clinical severity and modalities of treatment. They could show that the level of VNN1 expression paralleled disease severity. To get mechanistic insight, they generated the VIVA transgenic mouse model that overexpresses Vnn1 specifically in the gut epithelium thus mimicking the human pathological situation. The inventors show surprisingly that Vnn1 overexpression protects VIVA mice from colitis, by preserving colonocyte fitness and reinforcing several key features of the intestinal barrier. They demonstrate that the epithelial pantetheinase Vnn1 has a dual effect on colon: (1) its enzymatic products, cysteamine and pantothenic acid (vitamin B5) enhance coenzyme A regeneration and colon fitness through metabolic rewiring; (2) they favor microbiota-dependent accumulation of butyrate, previously shown to regulate mucosal energetics and to be reduced in IBD patients. The observed induction of Vnn1/VNN1 during colitis in mouse and human is therefore a compensatory mechanism. Under physiological conditions, food and microbiota provide an appropriate supply in cysteamine and pantothenate. The inventors believe that in severe IBD, the high induction of VNN1 is no longer protective because its enzymatic activity necessary for protection is not sufficiently supplied by lack of substrate. Importantly, the pharmacological administration of Vnn1 pantetheinase derivatives recapitulates this protection and its associated phenotypes in control C57BL/6 mice suggesting the possibility of reconditioning gut homeostasis. Therefore, the presence of increased VNN1 levels in IBD patients suggests that a vitamin B5-dependent pathway might be essential in the process of mucosal healing. This raises the necessity to investigate the need to maintain adequate vitamin B5 and CoA levels during the course and treatment of IBD.

The inventors demonstrated that co-administration of the products of Vnn1 activity (ie. Pan and CEA) to normal mice recapitulates most of the protective phenotypes conferred by the constitutive overexpression of Vnn1 in the VIVA model. Remarkably, oral administration of pantethine, the substrate of Vnn1 activity, leads to the same protective result.

### Therapeutic Methods and Uses

Accordingly, in a first aspect the present invention relates to a method for repairing intestinal mucosal barrier in subject in need thereof comprising administering to said subject an effective amount of a substrate of vanin-1 pantetheinase (Vnn1) and/or product(s) of vanin-1 pantetheinase (Vnn1) enzymatic activity.

As used herein, the term “vanin-1 pantetheinase”, also known as “Vnn1”, has its general meaning in the art and refers to an ubiquitous enzyme which hydrolyses D-pantetheine into cysteamine and pantothenate (vitamin B5) on the dissimilative pathway of CoA. Pantetheinase isoforms are encoded by the Vnn (vanin) genes and Vnn1 is the predominant tissue isoform in mice and humans.

In some embodiments, the substrate of vanin-1 pantetheinase is pantethine.

In some embodiments, the product(s) of vanin-1 pantetheinase enzymatic activity are pantothenate, pantothenate analog and/or cysteamine.

In some embodiments, the product(s) of vanin-1 pantetheinase enzymatic activity are pantothenate and cysteamine.

In some embodiments, the product(s) of vanin-1 pantetheinase enzymatic activity are pantothenate analog and cysteamine.

In other words, the present invention relates to a method for repairing intestinal mucosal barrier in subject in need thereof comprising administering to said subject an effective amount of an agent(s) of Vnn1 metabolism selected from the group consisting of i) pantethine, ii) pantothenate and/or pantothenate analog, iii) cysteamine, iv) pantothenate and cysteamine, and v) pantothenate analog and cysteamine.

As used herein, the term “pantethine” also known as “bis-pantethine” has its general meaning in the art and refers to a vitamin B5 analog having the following formula C₂₂H₄₂N₄O₈S₂. Its CAS number is 16816-67-4.

As used herein, the term “cysteamine” (CEA), also known as “2-aminoethanethiol” or by its brand name “Cystagon”, “Procysbi”, “Cystaran”, has its general meaning in the art and refers to a chemical compound having the following formula C₂H₇NS. Cysteamine is an amino thiol drug mainly used in the treatment of cystinosis. Its CAS number is 60-23-1.

As used herein, the term “pantothenate” also known as “pantothenic acid” (Pan), also known as “vitamin B5” has its general meaning in the art and refers to water-soluble B vitamin, an essential nutrient having the following formula C₉H₁₇NO₅. The IUPAC name is 3-[(2R)-(2,4-Dihydroxy-3,3-dimethylbutanoyl) amino]propanoic acid. Its CAS number is 599-54-2.

As used herein, the term “pantothenate analog” refers to a biologically active analog of pantothenic acid, that is converted into pantothenic acid in organism. Pantothenic acid analog include pantothenol such as dexpanthenol, the d-isomer of panthenol.

In some embodiments, the pantothenate analog is panthenol.

In some embodiments, the pantothenol is dexpanthenol.

In other word, the invention relates to i) a substrate of vanin-1 pantetheinase and/or product(s) of vanin-1 pantetheinase enzymatic activity for use for repairing intestinal mucosal barrier in subject in need thereof.

In other words, the invention relates to an agent(s) of Vnn1 metabolism selected from the group consisting of i) pantethine, ii) pantothenate and/or pantothenate analog, iii) cysteamine, iv) pantothenate and cysteamine, and v) pantothenate analog and cysteamine for use for repairing intestinal mucosal barrier in subject in need thereof.

In particular embodiment, the invention relates to pantothenate or pantothenate analog and cysteamine for use for repairing intestinal mucosal barrier in subject in need thereof.

In particular embodiment, the invention relates to cysteamine and pantothenate for simultaneous, separate or sequential for use for repairing intestinal mucosal barrier in subject in need thereof.

In particular embodiment, the invention relates to cysteamine and pantothenate analog such panthenol for simultaneous, separate or sequential for use for repairing intestinal mucosal barrier in subject in need thereof.

As used herein, the term “intestinal mucosal” or “intestinal mucosal barrier” has its general meaning in the art and refers to a critical barrier ensuring adequate containment of undesirable luminal contents within the intestine while preserving the ability to absorb nutrients. Intestinal mucosal barrier is a rapidly proliferating sheet of epithelial cells that sustains injury in response to stresses ranging from physiologic daily digestive trauma to severe insults associated with ischemia, chemicals, and infection. An intact mucosal barrier requires the adequate control of epithelial cell fitness, cell-junctions and specific functions such as the luminal secretion of mucus or anti-microbial peptides. Breaks in epithelial continuity impair mucosal barrier function, perturb normal absorptive and secretory transport properties, and render the host susceptible to local infection and distant organ pathology. The repair of intestinal mucosal injuries is a tightly regulated process involving epithelial restitution, cell proliferation and maturation, and the dedifferentiation of epithelial cells.

In some embodiments, the intestinal mucosal barrier is the colonic mucosal barrier.

As used herein, the term “subject” refers to any mammals, such as a rodent, a feline, a canine, and a primate. Particularly, in the present invention, the subject is a human afflicted with or susceptible to be afflicted with intestinal barrier defects. Particularly, in the present invention, the subject is a human afflicted with or susceptible to be afflicted with disease associated with intestinal barrier defects. Particularly, in the present invention, the subject is a human afflicted with or susceptible to be afflicted with inflammatory bowel diseases. In some embodiments, the subject has been treated with antibiotics and have developed intestinal mucosal barrier defects. Antibiotics are frequently used to cure infectious diseases, however, it may cause serious gastrointestinal dysfunction, such as mucosal barrier damages.

Intestinal barrier defects have been associated with a broad range of diseases, including gut inflammatory diseases (e.g. inflammatory bowel disease (IBD) and irritable bowel syndrome (IBS)), but also extra-intestinal disorders (e.g. liver disease such as chronic liver disease or alcoholic liver disease, diabetes such as type 1 diabetes or type 2 diabetes, obesity and obesity-related diseases, systemic infection, systemic lupus erythematosus, fungal or viral infection). For example, it is well known that disturbances in the intestinal barrier result in increased portal influx of bacteria or their products to the liver, where they cause or worsen a range of liver diseases. Indeed intestinal mucosal and vascular barrier is the functional and anatomical structure that serves as a playground for the interactions between the gut and the liver, limiting the systemic dissemination of microbes and toxins while allowing nutrients to access the circulation and to reach the liver as explained in Albillos A et al, J Hepatol. 2020 [60]. It is also well known that intestinal barrier defect can contribute to the to the development of the type 1 diabetes by causing diabetogenic antigens to enter the body tissues, contributing to beta-cell autoimmunity, as detailed in Mønsted MØ et al, J Autoimmun. 2021 [61] or Xia Li et al, Pediatr Diabetes [62]. 2015. A dysfunctional barrier is a common feature of obesity and type 2 diabetes, obersity-related complication are closely associated with altered intestinal barrier functions as reviewed in Allam-Ndoul et al, Int J Mol Sci. 2020 [63].

The method of the invention is thus particularly suitable to treat disease associated with intestinal barrier defects.

Thus, the invention refers to a method for treating disease associated with intestinal barrier defects in a subject in need thereof comprising administering to the subject a therapeutically effective amount of a substrate of vanin-1 pantetheinase and/or product(s) of vanin-1 pantetheinase enzymatic activity.

In other words, the invention refers to a method for treating disease associated with intestinal barrier defects in a subject in need thereof comprising administering to the subject a therapeutically effective amount of an agent(s) selected from the group consisting of i) pantethine, ii) pantothenate and/or pantothenate acid analog, iii) cysteamine, iv) pantothenate and cysteamine, and v) pantothenate acid analog and cysteamine

Thus, in some embodiments, the disease associated with intestinal barrier defects is inflammatory bowel disease, irritable bowel syndrome, liver disease such as chronic liver diabetes or alcoholic liver disease, diabetes such as type 1 diabetes or type 2 diabetes, obesity and obesity-related diseases, systemic infection, systemic lupus erythematosus, fungal or viral infection.

As used herein, the term “chronic liver disease” has its general meaning in the art and refers to diseases involving a process of progressive destruction and regeneration of the liver parenchyma leading to fibrosis and cirrhosis.

As used herein, the term “diabetes” has its general meaning in the art and refers to a group of metabolic disorders characterized by a high blood sugar level (hyperglycemia) over a prolonged period of time. Obesity has become a global epidemic and public health crisis, especially in last decades, and the incidence of obesity is continuing to rise at an alarming rate. Diabetes is classified into two main categories: type 1 diabetes and type 2 diabetes.

As used herein, the term “type 1 diabetes” has its general meaning in the art and refers to an autoimmune disease that is a form of diabetes in which very little or no insulin is produced by the islets of Langerhans (containing beta cells) in the pancreas. Increasing evidence, both functional and morphological, supports the concept of increased intestinal permeability as an intrinsic characteristic of type 1 diabetes (TID) in both humans and animal models of the disease [62, 63].

As used herein, the term “type 2 diabetes” has its general meaning in the art and refers to a form of diabetes that is characterized by a resistance to insulin and a loss of the ability to produce enough insulin in the pancreas.

As used herein, the term “obesity” has its general meaning in the art and refers to an abnormal or excessive fat accumulation that presents a risk to health. Obesity has been linked to increasing incidence of serious health risk factors and conditions. According to the invention, the obesity-related diseases includes but are not limited to insulin resistance, type 2 diabetes (T2D), nonalcoholic fatty liver disease (NAFLD) or atherosclerosis.

As used herein, the term “irritable bowel syndrome (IBS)” is a term for a variety of pathological conditions causing discomfort in the gastro-intestinal tract. It is a functional bowel disorder characterized by chronic abdominal pain, discomfort, bloating, and alteration of bowel habits in the absence of any organic cause. It also includes some forms of food-related visceral hypersensitivity, such as Gluten hypersensitivity.

As used herein the term “inflammatory bowel disease” has its general meaning in the art and refers to any inflammatory disease that affects the bowel. The term includes but is not limited to ulcerative colitis (UC), Crohn's disease (CD), especially Crohn's disease in a state that affect specifically the colon with or without ileitis, microscopic colitis (lymphocytic colitis and collagenous colitis), infectious colitis caused by bacteria or by virus, radiation colitis, ischemic colitis, pediatric colitis, pouchitis, celiac disease, undetermined colitis, and functional bowel disorders (described symptoms without evident anatomical abnormalities). CD and UC are chronic inflammatory diseases, and are not medically curable except for the use of surgery, although this may not eliminate extra-intestinal symptoms, and for CD, this does not preclude relapses. Accordingly, there is a medical need to specifically treat IBD patient with new therapeutic approach.

In some embodiments, the disease associated with intestinal barrier is inflammatory bowel disease (IBD).

In some embodiments, the inflammatory bowel disease (IBD) is crohn's disease (CD) or ulcerative colitis (UC).

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. According to the invention, the method of the invention allows to ameliorate the intestinal mucosal repair.

The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the terms “administering” or “administration” refer to the act of injecting or otherwise physically delivering a substance as it exists outside the body (e.g. pantethine and/or cysteamine and pantothenic acid) into the subject, such as by mucosal (such oral delivery), intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In some embodiments, a substrate of vanin-1 pantetheinase and/or product(s) of vanin-1 pantetheinase enzymatic activity (i.e the agent(s) of Vnn1 metabolism of the invention) are orally administered.

In some embodiments, a substrate of vanin-1 pantetheinase and/or product(s) of vanin-1 pantetheinase enzymatic activity (i.e the agent(s) of Vnn1 metabolism of the invention) are locally administered in the gut.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different

By a “therapeutically effective amount” is meant a sufficient amount of pantethine and/or cysteamine and pantothenic acid for the treatment of disease associated with intestinal barrier defects, such as IBD, at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the substrate of vanin-1 pantetheinase and/or product(s) of vanin-1 pantetheinase enzymatic activity (i.e the agent(s) of Vnn1 metabolism of the invention) are administered in combination with a therapeutic compound used to treat disease associated with intestinal barrier defects.

In some embodiments, the substrate of vanin-1 pantetheinase and/or product(s) of vanin-1 pantetheinase enzymatic activity (i.e the agent(s) of Vnn1 metabolism of the invention) are administered in combination with a therapeutic compound used to treat IBD or IBS.

In a particular embodiments, the therapeutic compound used to treat of gut inflammatory bowel disease are for example anti-TNF alpha compounds such as etanercept, infliximab and adalimumab; golimumab; certolizumab; vedolizumab; ustekinumab; onercept and CDP571 anti-inflammatory drugs such as corticosteroids, aminosalicylayes, mesalazines, balsalazide and olsalazine; immunosuppressant drugs such as azathioprine, mercaptopurine and methotrexate; antioxidants such as ascorbic acid, vitamin A, vitamin E; and antibiotics.

A used herein, the term “Anti-TNF alpha compounds” or “TNF alpha inhibitor” denotes all molecules which inhibit the activity and the expression of TNF-alpha.

As used herein, the term “Tumor necrosis factor alpha” also known as “TNF-alpha” or “TNF” has its general meaning in the art and refers to an inflammatory cytokine produced by macrophages/monocytes during acute inflammation and is responsible for a diverse range of signalling events within cells, leading to necrosis or apoptosis.

The anti-TNF alpha compounds can be a peptide, petptidomimetic, small organic molecule, antibody, siRNA or antisense oligonucleotide. Binding to TNF-alpha and inhibition of the biological activity of TNF-alpha may be determined by any competing assays well known in the art. For example, the assay may consist in determining the ability of the agent to be tested as inhibitor of TNF-alpha to bind to TNF-alpha. The binding ability is reflected by the Kd measurement. Test to identify an anti-TNF alpha compound are well known and described in the art (see Zia et al, Identification of potential TNF-α inhibitors: from in silico to in vitro studies. Nature. 2020 [64]). For example anti-TNF alpha therapy to treat disease associated with intestinal barrier defects such as inflammatory bowel disease are well known in the art and include anti-TNF-alpha antibodies, antisense oligonucleotides, microRNA, small interfering RNA as described in Gareb et al, Pharmaceutics, 2020 [65].

As used herein, the term “peptidomimetic” refers to a small protein-like chain designed to mimic a peptide.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

As used herein, the term “antibody” refers to an immunoglobulin molecules and immunologically active portions of immunoglobulin molecules, i.e., molecules that contain an antigen binding site that immunospecifically binds an antigen. As such, the term antibody encompasses not only whole antibody molecules, but also antibody antibody fragments comprising an antigen-binding domain (such as Fab, Fab′ and F (ab) 2, scFv, the fragments comprising either a VL domain or a VH domain), monoclonal antibodies, polyclonal antibodies, chimeric antibodies, humanized antibodies, primary antibodies, monospecific antibodies, multi-specific antibodies, single-chain antibodies (eg of camelid type). The antibodies according to the invention may be antibodies of any type, for example, IgG, IgE, IgM, IgD, IgA and IgY, of any class, for example, IgG1, IgG2, IgG3, IgG4, IgA1 and IgA2 or any subclass.

In particular embodiments, the anti-TNF alpha compound is an anti-TNF alpha antibody

In particular embodiments, the anti-TNF alpha compound is selected from the group consisting of etanercept, infliximab and adalimumab; golimumab; certolizumab; vedolizumab; ustekinumab; onercept, CDP571, AVX-470, ISIS 25302 and ISIS 25302.

As used herein, the terms “combined treatment”, “combined therapy” or “therapy combination” refer to a treatment that uses more than one medication. The combined therapy may be dual therapy, bi-therapy or tri-therapy.

The medications used in the combined treatment according to the invention are administered to the subject simultaneously, separately or sequentially.

Thus, the invention refers to a method for treating disease associated with intestinal barrier defects in a subject in need thereof comprising administering to the subject a therapeutically effective amount of a substrate of vanin-1 pantetheinase and/or product(s) of vanin-1 pantetheinase enzymatic activity in combination with a therapeutic compound used to treat disease associated with intestinal barrier defects.

In particular, the invention refers to a method for treating inflammatory bowel disease in a subject in need thereof comprising administering to the subject a therapeutically effective amount of a substrate of vanin-1 pantetheinase and/or product(s) of vanin-1 pantetheinase enzymatic activity in combination with anti-TNF alpha compound.

In other words, the invention refers to a method for treating disease associated with intestinal barrier defects in a subject in need thereof comprising administering to the subject a therapeutically effective amount of an agent(s) of Vnn1 metabolism selected from the group consisting of i) pantethine, ii) pantothenate and/or pantothenate analog, iii) cysteamine, iv) pantothenate and cysteamine, and v) pantothenate analog and cysteamine; in combination with a therapeutic compound used to treat disease associated with intestinal barrier defects.

In other words, the invention refers to a method for treating inflammatory bowel disease in a subject in need thereof comprising administering to the subject a therapeutically effective amount of agent(s) of Vnn1 metabolism selected from the group consisting of i) pantethine, ii) pantothenate and/or pantothenate analog, iii) cysteamine, iv) pantothenate and cysteamine, and v) pantothenate analog and cysteamine; in combination with a therapeutic compound used to treat disease associated with intestinal barrier defects.

### Pharmaceutical Composition

The substrate of vanin-1 pantetheinase and/or product(s) of vanin-1 pantetheinase enzymatic activity may be combined with pharmaceutically acceptable excipients, and optionally sustained-release matrices, such as biodegradable polymers, to form therapeutic compositions.

Accordingly, in a second aspect, the invention relates to a pharmaceutical composition comprising the substrate of vanin-1 pantetheinase and/or product(s) of vanin-1 pantetheinase enzymatic activity for use for repairing intestinal mucosal barrier in subject in need thereof.

The invention also relates to a pharmaceutical composition comprising the substrate of vanin-1 pantetheinase and/or product(s) of vanin-1 pantetheinase enzymatic activity for use for treating disease associated with intestinal barrier defects in a subject in need thereof.

In other words, the invention relates to a pharmaceutical composition comprising an agent(s) of Vnn1 metabolism selected from the group consisting of i) pantethine, ii) pantothenate and/or pathotenate analog, iii) cysteamine, iv) pantothenate and cysteamine, and v) pantothenate analog and cysteamine for use for repairing intestinal mucosal barrier in subject in need thereof.

In other words, the invention relates to a pharmaceutical composition comprising an agent(s) of Vnn1 metabolism selected from the group consisting of i) pantethine, ii) pantothenate and/or pantothenate analog, iii) cysteamine, iv) pantothenate and cysteamine, and v) pantothenate analog and cysteamine for use for treating disease associated with intestinal barrier defects in a subject in need thereof.

As used herein, the term “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising inhibitors of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

### Method for Diagnosing and Monitoring

In the management of patients with inflammatory bowel diseases (IBD), there is also a need to identify prognostic markers. Surprisingly, extended metabolomic analysis of fecal samples using liquid chromatography-mass spectrometry (LC-MS) technology documented high levels of indoxyl sulfate (IS) in the feces of VIVA and CEA+Pan treated WT mice (FIG. 1C). The inventors confirmed the presence of elevated levels of IS in the urine of VIVA mice (FIG. 1A). They also found significantly augmented urine levels of IS in IBD patients compared to healthy patients (FIG. 1B).

Accordingly, in a third aspect, the invention refers to an in vitro method for diagnosing inflammatory bowel disease in a subject, comprising the steps of i) determining in a sample obtained from a subject, the expression level of indoxyl sulfate; ii) comparing the expression level of indoxyl sulfate determined at step i) with a reference value; and iii) concluding that the subject has inflammatory bowel disease when the expression level of indoxyl sulfate determined at step i) are higher than the reference value.

In some embodiments, inflammatory bowel disease is crohn's disease (CD) or ulcerative colitis (UC).

As used herein and according to all aspects of the invention, the term “sample’ denotes feces sample and urinary sample. In particular embodiment, the sample is an urinary sample.

Thus, the invention refers to an in vitro method for diagnosing inflammatory bowel disease in a subject, comprising the steps of i) determining in a urinary sample obtained from a subject, the expression level of indoxyl sulfate; ii) comparing the expression level of indoxyl sulfate determined at step i) with a reference value; and iii) concluding that the subject has inflammatory bowel disease when the expression level of indoxyl sulfate determined at step i) are higher than the reference value.

As used herein, the term “indoxyl sulfate” (IS), also known as “3-indoxylsufalte” or “3-indoxylsulfuric acid” has its general meaning in the art and refers to a metabolite of dietary 1-tryptophan that acts as a cardiotoxin and uremic toxin. Its CAS number is 487-94-5.

As used herein, the term “expression level” indicates the quantity or concentration of the metabolite of interest (i.e indoxyl sulfate). In some embodiments, the “expression level” means the quantitative measurement of the metabolite expression relative to a negative control.

Typically indoxyl sulfate expression level may be measured for example by capillary electrophoresis-mass spectroscopy technique (CE-MS), flow cytometry, mass cytometry, or ELISA performed on the sample.

Such methods comprise contacting a sample with a binding partner capable of selectively interacting with proteins present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS).etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Particularly, a mass spectrometry-based quantification methods may be used. Mass spectrometry-based quantification methods may be performed using either labelled or unlabelled approaches [DeSouza and Siu, 2012]. Mass spectrometry-based quantification methods may be performed using chemical labeling, metabolic labeling or proteolytic labeling. Mass spectrometry-based quantification methods may be performed using mass spectrometry label free quantification, a quantification based on extracted ion chromatogram (EIC) and then profile alignment to determine differential level of polypeptides.

Particularly, a mass spectrometry-based quantification method particularly useful can be the use of targeted mass spectrometry methods as selected reaction monitoring (SRM), multiple reaction monitoring (MRM), parallel reaction monitoring (PRM), data independent acquisition (DIA) and sequential window acquisition of all theoretical mass spectra (SWATH) [Moving target Zeliadt N 2014 The Scientist;Liebler Zimmerman Biochemistry 2013 targeted quantitation pf proteins by mass spectrometry; Gallien Domon 2015 Detection and quantification of proteins in clinical samples using high resolution mass spectrometry. Methods v81 p15-23; Sajic, Liu, Aebersold, 2015 Using data-independent, high-resolution mass spectrometry in protein biomarker research: perspectives and clinical applications. Proteomics Clin Appl v9 p 307-21].

Particularly, the mass spectrometry-based quantification method can be the mass cytometry also known as cytometry by time of flight (CYTOF) (Bandura DR, Analytical chemistry, 2009).

Particularly, the mass spectrometry-based quantification is used to do peptide and/or protein profiling can be use with matrix-assisted laser desorption/ionisation time of flight (MALDI-TOF), surface-enhanced laser desorption/ionization time of flight (SELDI-TOF; CLINPROT) and MALDI Biotyper apparatus [Solassol, Jacot, Lhermitte, Boulle, Maudelonde, Mangé 2006 Clinical proteomics and mass spectrometry profiling for cancer detection. Journal: Expert Review of Proteomics V3, 13, p311-320; FDA K130831].

Colorimetry or colourimetry is a technique used to determine the concentration of colored compounds in solution. A colorimeter is a device used to test the concentration of a solution by measuring its absorbance of a specific wavelength of light. Particularly, the colorimetry can be based on the Curzon and Walsh method where the indoxyl sulfate react with a chromogen as disclosed in Curzon et al. Clin Chim Acta. 1962,

Methods of the invention may comprise a step consisting of comparing the metabolites and fragments concentration in circulating cells with a control value. Typically, a level of a metabolite can be expressed as nanograms per microgram of tissue or nanograms per milliliter of a culture medium, for example. Alternatively, relative units can be employed to describe a concentration. Expression levels may be expressed as absolute expression level or normalized expression level of indoxyl sulfate.

In some embodiments, the reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the score in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the score in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of a) providing a collection of samples; b) providing, for each sample provided at step a), information relating to the actual clinical outcome for the corresponding subject (i.e. having IBD); c) providing a serial of arbitrary quantification values; d) determining the expression level of indoxyl sulfate for each sample contained in the collection provided at step a) so as to calculate the score as described above; e) classifying said samples in two groups for one specific arbitrary quantification value provided at step c), respectively: (i) a first group comprising samples that exhibit a quantification value for the score that is lower than the said arbitrary quantification value contained in the said serial of quantification values; (ii) a second group comprising samples that exhibit a quantification value for said score that is higher than the said arbitrary quantification value contained in the said serial of quantification values; whereby two groups of samples are obtained for the said specific quantification value, wherein the samples of each group are separately enumerated; f) calculating the statistical significance between (i) the quantification value obtained at step e) and (ii) the actual clinical outcome of the patients from which samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g).

For example the score has been assessed for 100 samples of 100 patients. The 100 samples are ranked according to the determined score. Sample 1 has the highest score and sample 100 has the lowest score. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the score corresponding to the boundary between both subsets for which the p value is minimum is considered as the predetermined reference value.

In some embodiments, when the subject is diagnosed with inflammatory bowel disease according to the method of the invention, the method for treating IBD according to the invention is performed.

Thus, the invention refers to a method for treating inflammatory bowel disease in a subject in need thereof, comprising the step of i) determining if the subject have inflammatory bowel disease according to the method of the invention and ii) administering a substrate of vanin-1 pantetheinase and/or product(s) of vanin-1 pantetheinase enzymatic activity when the subject is determined having inflammatory bowel disease.

In other words, the invention refers to a method for treating inflammatory bowel disease in a subject in need thereof, comprising the step of i) determining if the subject have inflammatory bowel disease according to the method of the invention and ii) administering an agent(s) of Vnn1 metabolism selected from the group consisting of i) pantethine, ii) pantothenate and/or pantothenate analog, iii) cysteamine, iv) pantothenate and cysteamine, and v) pantothenate analog and cysteamine when the subject is determined having inflammatory bowel disease.

In some embodiments, the substrate of vanin-1 pantetheinase and/or product(s) of vanin-1 pantetheinase enzymatic activity (i.e the agent(s) of Vnn1 metabolism of the invention) is administered in combination with a therapeutic compound used to treat inflammatory bowel disease.

The invention will be further illustrated by the following figures and examples. However, these examples and figures should not be interpreted in any way as limiting the scope of the present invention.

### FIGURES

FIG. 1. Overexpression of Vnn1 on gut epithelium confers changes in the microbiota composition and leads to augmented fecal levels of indoxyl sulfate (IS). (A) Dosage of indoxyl sulfate in the urine of WT and VIVA mice. (B) Comparative measure of urinary concentrations of indoxyl sulfate in IBD patients (mixed CD and UC population) versus healthy controls. (C) Augmented levels of Indoxyl sulfate (LC-MS analysis) in feces from VIVA mice.

FIG. 2. Supplementation of WT mice with Vnn1-pantetheinase substrate or products recapitulates the VIVA phenotype. (A) Upper panel: diagram representing the protocol used for the combined treatment with Pantothenic acid (Pan) plus cysteamine (CEA), and induction DSS-colitis in WT mice. Lower panel: Monitoring of the total body weight loss in pre-treated (CEA+Pan) or non-treated control (PBS) WT mice during DSS-colitis (n=20). (B) Measurement of colon shortening at day 11 of the DSS-colitis protocol (n=5). (C) Mucosal permeability assessed by scoring of the level of FITC-labeled dextran retrieved in blood after gavage of mice at day 4 of DSS (n=5). (D) Significant variations in the repartition of some microbiota bacterial strains between non-treated (WT) and CEA+Pan-treated (C+P) mice (n=10). (E) Calculated butyrate over acetate ratio over time during CEA+Pan treatment showing a progressive increased in butyrate levels in feces (n=14). (F) Upper panel: diagram representing the protocol used for the treatment with Pantethine and induction DSS-colitis in WT mice. Lower panel: monitoring of weight loss in Pantethine-treated and untreated mice (PBS) during DSS-colitis (n=5). (G) Upper panel: monitoring of weight loss in WT and VIVA mice during repeated cycle of low DSS (0.75%) (n=5). Lower panel: dosage by ELISA of Vnn1 in feces during the DSS cycles (n=5).

FIG. 3. Treatment with Cysteamine+Pantothenate protects WT mice to severe DSS-colitis. Administration of pantethine has no effect on Vnn1 KO mice. (A-B) Respective effect of the single or combine administration of Cysteamine and Pantothenate. (D) CEA+PANT treatment affect the resilience of the microbiota.

FIG. 4. Effect of the CEA+Pan treatment on TNBS-colits. A. Monitoring of the total body weight loss in pretreated (CEA+Pan) or non-treated WT mice during 2,4,6-trinitrobenzene sulfonic acid (TNBS)-colitis. Control mice received intrarectal administration of EtOH/H2O. B. Macroscopic assessment of the colitis grade was scored according to a previously reported scoring system

FIG. 5: Epithelial restitution after exposure to TNF in colon organoids from WT and VIVA mice, and treated or not with panthetine.

### EXAMPLE

### Material & Methods

### IBD) Biobank and RNAseq Studies.

Biopsies samples from IBD patients were collected at the department of gastroenterology directed by Pr. Grimaud at APHM HOPITAL NORD, Marseille (France). The study protocol was approved by the institution's Ethics Committee (ClinicalTrials.gov Identifier: NCT02304666). RNA extraction from colonic biopsies was performed using the Qiagen AllPrep RNA/DNA Mini Kit. RNA integrity was measure by AGILENT bioanalyzer. RNAseq process was carried out at the plateform GenomEast IGBMC, ILLKRICH, France. (France). All samples were sequenced in 50-length Single-Read. Reads were mapped onto the hg38 assembly of the Homo sapiens genome using Tophat 2.0.14 and bowtie version 2-2.1.0. Only uniquely mapped reads have been retained for further analyses. Quantification of gene expression has been performed using HTSeq-0.6.1 with annotations coming from Ensembl 87. Read counts have then been normalized across libraries with the median-of-ratios method proposed by Anders and Huber. To check if the normalization was correctly performed, Relative Log Expression (RLE) plots were drawn. Genes with less than 10 counts in the sum of all samples were discarded for downstream analyses. Counts were scaled by trimmed mean of M-values (https://doi.org/10.1093/bioinformatics/btp616) for EGSEA (https://doi.org/10.1093/bioinformatics/btw623), or normalized by the pseudolog2 of the variance-stabilizing transformation (https://doi.org/10.1186/s13059-014-0550-8) for WGCNA (https://doi.org/10.1186/1471-2105-9-559), accordingly to their respective guidelines.

### Data Access

All raw and processed sequencing data generated in this study have been submitted to the NCBI Gene Expression Omnibus (GEO) under the meta-serie GSE174159 (https://www.ncbi.nlm.nih.gov/geo/query/acc.cgi?acc=GSE174166). Scripts used in this study are available in the GitHub repository (https://github.com/guillaumecharbonnier/mw-{thisArticleFirstAuthorName}2021).

### Animal Care and Treatment.

Mice were kept in a specific pathogen-free mouse facility at the CIML. All experiments were carried on 8-17 weeks-old female VIVA mice or wild type C57BL/6 controls either bred at the CIML or purchased from Janvier. Experiments were performed in accordance with institutional guidelines for animal care and use. This experimental design was authorized by the Ethical Committee for Animal Experimentation (no. 02820.01). For drug treatments before colitis induction, mice were intraperitoneally injected every 2 days for two weeks with Cysteamine hydrochloride (120 mg/kg) and Pantothenate (500 mg/kg/) or D-Pantehine (1 g/Kg). All reagents were purchased from Sigma-Aldrich. Oral administration of D-Pantethine was done by gavage every 2 days at a dose of 150 mg/Kg. For microbiota depletion, mice received by gavage a daily dose of 200 μL of a broad-spectrum antibiotic cocktail consisting of Metronidazole (1 mg/ml), Vancomycin (0.5 mg/ml), Gentamycin (1 mg/ml), Neomycin (1 mg/ml), and Ampicillin (1 mg/ml) for 10 days.

### Generation of the VIVA Mouse.

The pBluescript II KS Villin MES SV40 polyA plasmid containing 9 kb of the murine Villin regulatory regions and a multiple cloning region (MCS) at its 3′ side was kindly provided by Dr. Sylvie Robine (UMR144, Institut Curie, Paris). 1539bp of the Vnn1 complete cDNA coding sequences were inserted into the MCS using the MluI-BsIWI restriction sites (suppl.data). The linearized 10853bp SalI-digested Villin-Vnn1 transgene was purified using the QIaquick gel extraction kit (Qiagen). The transgene was then injected in fertilized oocytes pronuclei from C57BL/6xCBA/j hybrid mice. Transgene transmission to germinal cells was verified by a PCR strategy using primers encompassing the 793bp long intron 3-4 segment. Afterward mice were backcrossed for 9 generations on the C57BL/6 background. Expression of the transgene in the mouse organs was monitored by qRT-PCR and by ELISA, and the pantetheinase activity in tissue was measured. VIVA+/− mice with the highest fecal pantetheinase activity were crossed to obtain the VIVA homozygous mice.

### DSS-Colitis Induction and Scoring.

Acute colitis was induced by a 7-day oral administration of 2% DSS (MP Biomedical) in the drinking water. In the chronic colitis model, mice were treated with 0.75% DSS during 2 weeks, then water for 15 days followed by a second exposition to 0.75% DSS for 2 weeks. Colons were removed, measured, longitudinally cut and coiled with the mucosal layer outwards, then fixed with formal, and embedded in paraffin. Swiss rolls were sectioned at 3.5 μm and stained with haematoxylin and eosin (HE). Histological scores were blindly assessed by a pathologist following the criteria described in suppl.data Table S1.

### Histological Studies.

Vnn1 and Ep-CAM expression was analyzed on cryosections using the purified 407 (Santa-Cruz) and Ep-CAM antibodies (Epitomics). A Cy™3 AffiniPure Donkey Anti-Rat and an Alexa Fluor® 488 Donkey Anti-Rabbit IgG (H+L) were used respectively as secondary antibodies. Slides were mounted in Prolong Gold with dapi (Invitrogen) and observed with a Zeiss LSM 510 confocal microscope (Carl Zeiss, Jena, Germany).

Paraffin sections were prepared to monitor epithelial cell proliferation and the neutrophil infiltration using an anti-Ki67 antibody (BD Bioscience) and anti-Ly6G antibody (BD Pharmingen), respectively. ImmPRESS® HRP Goat Anti-Rat IgG Polymer Detection Kit, Peroxidase and DAB from Vector Laboratories were used to reveal the staining. For cell counting 5 pictures were taken per swiss-roll and cells were counted using the ImageJ software and cell count plugin. A standard area was taken for each pictures and Ki67− and Ly6G+ cells were counted for each standard area.

Mucosal polysaccharides and goblet cells were detected after Periodic Acid-Schiff (PAS) staining of the sections. Colons were fixed in Carnoy's solution then embedded in paraffin. Slides were incubated 5 min in a solution of periodic acid 0.5%, rinsed with water and incubated 15 min in a solution of Schiff. After rinsing with warm water for 5 min, slides were incubated with Hematoxylin 5 sec, rinsed and mounted in DePeX mounting medium (SERVA). PAS positive cells were counted along 50 full size longitudinal crypts on WT and VIVA sections obtained from 3 different animals per genotype.

A specific anti-Muc2 antibody from (Novusbio) was used to detect Mucin-2 by IF, using a Cy™3 AffiniPure Donkey Anti-Rabbit IgG (H+L) as secondary antibody. Quantification of Muc-2 labelling was done using the ImageJ software by measuring the mean of fluorescence of Muc2 on total tissue area on 10 colon sections per mouse, and 3 different mice per genotype.

### Gene Expression.

Total mRNA from tissues or cells was purified using the RNeasy Mini Kit (Qiagen). For qRT-PCR analysis, 0.5-1 μg RNA was reverse transcribed with the SuperScript II RT kit (Life Technologies). Amplification was performed on a 7500 Fast Real Time PCR system (Applied Biosystems) using SYBR green Master Mix (Takara) and specific primer pairs (Table S2). Expression levels were normalized to the control gene actin. For the expression of genes encoding anti-microbial peptides in isolated colonocytes, samples were normalized using the expression of the SPDEF gene as a marker of goblet cell enrichment in the cell preparation.

### Flow Cytometry.

Mice were euthanized at day 4 of the DSS treatment and colons were recovered, washed with ice cold PBS, cut into small pieces, washed again with EDTA-HBSS several times, and digested with Collagenase VIII from Clostridium histolyticum (Sigma-Aldrich) to obtain single cell suspensions. Cells were recovered using a Percoll gradient and stained with specific antibodies: Cd11b (M1/7); CD64 (X54-5/7.1); CD3 (17A2); NK1.1 (PK136); CD19 (6D5); Ly6G (1A8); Ly6C (Al-21); CD45 (104); CD24; CD11c (N418) and SYTOX Green Nucleic Acid Stain (Thermo Fisher Scientific). The labelled cells were processed using a LSR II flow cytometer and the data were analyzed using the FACSDIVA software (BD Biosciences). Lamina propria CD64+ gut monocytes were gated in CD11b+; CD24−; CD45+; Lin− live cells. Ly6G+ neutrophils were gated in CD45+; CCD11b+; CD11c+; CD45+ live cells.

### Epithelial Permeability.

Fluorescein isothiocyanate-dextran (Sigma-Aldrich) was administered to mice at 44 mg/100 g body weight by oral gavage. 5 hours after gavage, mice were anesthetized with 3% isoflurane and blood was recovered by retro orbital puncture. Plasma FITC fluorescence was measured using a TECAN Infinite M 1000 PRO microplate reader.

Electron Microscopy Analysis.

Samples were prepared using the NCMIR protocol for serial blockface scanning electron microscopy (West et al, 2010). 70-nm ultrathin sections were performed on a Leica UCT Ultramicrotome (Leica) and deposited on formvar-coated slot grids. The grids were observed in an FEI Tecnai G2 at 200 KeV, and acquisition was performed on a Veleta camera (Olympus). The size of the apical mucus layer was measured on 17 images per mouse and 3 mice per genotype. The mucus accumulation and the detection of invading bacteria at the bottom of the crypts were investigated as indicated in FIG. 3I in 3 different mice per genotype.

CoA Extraction and Quantification from Mouse Colons.

Colons were removed and flushed in HBSS-0.25% BSA containing a cocktail of phosphatase inhibitors (100 μM suramin; 100 μM levamisole and 1 mM NaF). Tissues were lyophilized, frozen in liquid nitrogen and dried on a SpeedVac system for 2 hours to remove any residual moisture. Each dried whole colon was then ground into a fine powder of which 20 mg was weighed and subjected to extraction. Tissue extraction was performed as a modified method as previously described by Shurubor et al. To each 20 mg of ground colon 200 μl of ice-cold 5% perchloric acid (in dH2O) containing 50 μM tris (2-carboxyethyl) phosphine (TCEP) was added after which the samples were kept on ice and periodically vortexed for 15 seconds every 2-3 minutes over a 15-20 minute period. In between vortexing steps the cells were subjected to preliminary homogenization by grinding with a metal spatula. Subsequently the cells were fully homogenized by sonication for 25 seconds in an ultrasonic water bath at full power. Samples were then put on ice and subjected to another round of vortexing as described above to allow for optimal extraction of CoA. Extracts were then centrifuged for 10 minutes at 13000×g to remove cell debris after which 100 μl of the supernatant was removed and filtered with a 0.2 μm syringe filter (PALL Acrodisc, 13 mm). 50 μl of the filtered extract was then neutralized by adding about 45 μl 1 M NaOH until a pH of 6.5-7.0 was reached. 30 μl neutralized extract was then added to 36.85 μl derivatization mixture containing 68 μM TCEP, 50 mM TRIS (pH 6.8) and 17.85 μl 100% acetonitrile. Samples were then incubated for 15 minutes to fully reduce any disulphides. Finally 3.15 μl of 10 mM N-[4-(7-diethylamino-4-methyl-3-coumarinyl)phenyl]maleimide (CPM, in 100% acetonitrile) was added to bring the total volume up to 70 μl and the total acetonitrile concentration to 30%. CoA levels of each sample was then analyzed via HPLC and quantified as described [60].

### In Vivo Sunset and Investigation of Protein Translation

One dose (40 nmol/g) of puromycin (Sigma-Aldrich) was administered intraperitoneally to the mice. Exactly 30 min after injection, colons were extracted, flushed in PBS and frozen in liquid N2 for Western-Blot analysis or snap frozen in Tissue-Tek OCT compound (Sakura Finetek Europe) for IF analysis. Thereafter, cryosections were fixed and permeabilized during 30 min with the Fixation/Permeabilization Solution Kit (BD biosciences), then incubated 1 h30 at RT with the Anti-Puromycin AF488 antibody (Millipore) diluted in Perm/Wash Buffer and counterstained with dapi. For Western Blot analysis, frozen tissues were homogenized for 20 s in ice-cold RIPA buffer (25 mM Tris, pH 7.6; 150 mM NaCl; 1% NP40; 1% DOC; 0.1% SDS, proteases inhibitors cocktail). The whole homogenate was centrifuged at 10000 rpm for 10 min at 4° C. and protein concentration in the supernatant was determined with BCA assay. Equivalent amounts of protein (50 μg) from each sample were dissolved in Laemmli buffer and subjected to electrophoretic separation on 10% SDS-PAGE acrylamide gels. Proteins were transferred to a PVDF membrane and stained with Ponceau S to verify equal loading in all lanes. After washing in water, membranes were blocked with 5% BSA in PBS-0.5% Tween for 1 h followed by an overnight incubation at 4° C. with mouse IgG2a monoclonal anti-puromycin antibody (Millipore) dissolved in PBS containing 5% BSA. Membranes were washed for 15 min in PBST and then incubated for 1 h at room temperature in 5% BSA-PBS containing horseradish peroxidase conjugated anti-mouse IgG Fc antibody (Sigma-Aldrich). After 15 min of washing in PBST, the blots were developed with enhanced chemiluminescence (ECL) reagent (Pierce; ThermoFisherScientific, Rockford, IL, USA). Densitometric measurements were performed by determining the density of each whole lane with ImageJ. A Ponceau S staining was done to verify equal loading for each sample.

### Preparation of Isolated Mice Colonocytes.

The colons, from the caecum to the rectal ampulla, were rapidly removed and flushed clear with calcium free HBSS. Each colon was everted, ligated at the proximal and distal ends, then placed in a tube containing 10 ml of HBSS-0.25% BSA 5 mM EDTA. After an incubation for 30 minutes at 37° C. in a shaker, the colons were rinsed in fresh calcium-free HBSS to remove excess EDTA and placed in a new tube containing 10 ml of HBSS-0.25% BSA. Vigorous manual stirring for two minutes readily disaggregated colonocytes, which were then separated by centrifugation at 500 g for 2 min at 4° C. The cells were twice washed in PBS and used for IF (Mitosox), lactate quantification, or qRT-PCR.

### Cell Culture.

Human intestinal Caco-2 cells were maintained in DMEM containing 10% FBS, 1% sodium pyruvate, 1% non-essential amino acids and 1% penicillin-streptomycin. To induce differentiation and polarization, Caco-2 cells were grown in 24-well plate and permeable PET membrane filter supports (Transwell 0.4 μM pore size). Culture medium was changed three times a week and experiments were conducted on days 17-21 post-seeding. Treatment with fecal filtrate was added in the apical medium and after 16 hr of treatment Caco-2 cell extracts were collected for qRT-PCR.

### MitoSox Mito Tracker

Colonocytes were seeded in chambered cover glass (Lab-Tek Nunc) coated with Cell-Tak (Corning) while loaded with 50 nM MitoTracker Green FM reagent and 1 μM MitoSox Red in HBSS/Ca/Mg, a mitochondrial superoxide indicator. The both reagents were purchased by Invitrogen. After an incubation of 15 min at 37° C., cells were washed in warm buffer and viewed with a confocal microscope.

Caco-2 cells seeded in Lab-Tek in a volume of 0.4 ml were treated with 40 μl of fecal extract filtrate, Antimycin A (Sigma-Aldrich) at 20 μM or sodium butyrate 1 mM. 24 hrs after incubation cells were washed and loaded with MitoTracker reagents.

### Lactate Quantification.

Lactate concentrations were quantified according to the manufacturer's protocols (Sigma-Aldrich). Briefly, colonocytes were resuspended in four volumes of lactate assay buffer, sonicated 10 cycles and centrifuged at 13,000 g for 10 min to remove insoluble material. Samples were deproteinized with a 10-kD molecular weight cut-off spin filter. A master reaction mix containing 46 μl lactate assay buffer, 2 μl lactate enzyme mix, and 2 μl lactate probe was added on 50 μl sample solution. Reactions were incubated at RT for 30 min and sample absorbance measured at 570 nm on a microplate reader.

### Laser Capture Microdissection (LCM) and Transcriptomic Analysis.

After 4 days of DSS administration mice were sacrificed and a 1 cm segment of the distal colon was recovered and frozen embedded in OCT. HE stained 5 μm colon sections were used to capture the epithelial cell layer from the colonic mucosa using the ArcturusXT Laser Capture Microdissection (LCM) System (Applied Biosystems) following manufacturer's procedures. Total RNA was afterwards extracted from purified epithelial mucosal cells using the Arcturus PicoPure RNA Isolation kit (Applied Biosystems). RNAs samples were validated by Agilent 2001 bioanalyzer (Agilent Technologies) and then used for hydridization in microarray studies using Affymetrix genchip mouse genome arrays (platform GenomEast IGBMC, ILLKRICH, France). Analysis of microarray data was performed by the Bioinformatic platform at CIML. Briefly, the mouse Gene 1.0 ST CEL files corresponding to WT (n=5) and ViVA (n=5) mice treated or not with DSS, were pre-processed through Bioconductor in the R statistical environment (version 3.2.2). Quality control of the array hybridization (NUSE and RLE plot) and normalization of the raw Affymetrix expression data with Robust Multi-array Average (Irizarry et al.2003, Biostatistics) were performed using the oligo package. Differentially expressed genes (DEG) between DSS-treated and non-treated mice in a statistically significant manner were selected as having an FDR (Benjamini-Hochberg correction)<0.05 after empirical Bayes processing using the Limma package. These DEGs were used as genesets for the BubbleMap analysis (Spinelli et al.2015, BMC Genomics) where each bubble is a GSEA result and summarizes the information from the corresponding enrichment plot. GSEA was also performed in order to extract the leading edges corresponding to the BubbleMap enrichments of the DEGs identified in one genotype against the corresponding response obtained in the other genotype. Selection of the genes overlapped in leading edges were submitted to Enrichr software.

### Microbiota Analysis.

16S rRNA gene sequencing and analysis Genomic DNA from feces was extracted with Mobio power soil DNA isolation kit. 16S rRNA gene variable region (V4) was amplified using barcoded (12 base) primers to tag the individual samples and sequenced using standard methods on an Illumina Miseq instrument. Sequence analysis was performed in Mothur. Metagenomic contents of microbial communities were predicted from 16S rRNA gene profiles by implementing PICRUSt. Sequences with at least 97% similarity were clustered in into species Operational Taxonomical Units (OTUs) using neighbor joining algorithm within mothur. OTU abundance were normalised by subsampling to the lowest number of sequences within analysed groups. Percent abundance of OTUs was log transformed to perform Principal Coordinate Analysis (PCoA).

### Fecal Extracts Preparation.

100 mg mice feces were homogenized in 1 ml of Phosphate buffer saline containing 50 μg/ml gentamycin, 100 Units/ml penicillin and 100 μg/ml streptomycin. The homogenate was centrifuged 15 min 10000 rpm at 4° C. Then, the supernatant was filtrated on a Nanosep column with 3K Omega (Pall) and the eluate used immediately to treat the cells.

### NMR Metabolomics.

Aqueous fecal extracts were prepared with 30 mg of thawed feces in 1 ml of phosphate buffer saline (1.9 mM Na2HPO4, 8.1 mM NaH2PO4, 150 mM NaCl, pH 7.4) containing 90:10 D2O/H2O (v/v) for the field lock of the NMR spectrometer and 1 mM of sodium 3-(trimethylsilyl) [2,2,3,3,-2H4] propionate (TSP) acting as a chemical shift reference. The mixture was homogenized and sonicated to destroy bacterial cells. The samples were centrifuged at 4° C. for 1 h at 18000×g. Supernatants were collected and centrifuged at 4° C. for 15 min at 15000×g. Then, 600 μl of supernatant were transferred into 5 mm NMR tubes or stored at −80° C. until analysis.

All NMR experiments were carried out on a Bruker Avance III spectrometer operating at 600 MHz for the 1 H frequency equipped with a TXI triple resonance probe and a Sample Jet autosampler which enables the storage of 5 racks of 96 NMR tubes at 5° C. The sample temperature was controlled at 300 K during experiments. Spectra were recorded using a Carr-Purcell-Meiboom-Gill (cpmgpr1d) NMR spin echo sequence [90°-(τ-180°-τ)n] with a total spin echo time of 64 ms and a number of loops of 200, preceded by a water presaturation pulse during a relaxation delay of 2 s to reduce the signal intensities of lipids and macromolecules. For each spectrum, 128 free induction decays (FID) of 65 k data points were collected using a spectral width of 20 ppm with an acquisition time of 2.72 s. For all the spectra, the FIDs were then multiplied by an exponential weighting function corresponding to a line broadening of 0.3

Hz and zero-filled prior to Fourier transformation. The spectra were automatically phased and baseline corrected and referenced to the internal standard (TSP; δ=0.0 ppm). Assignments of the fecal metabolite signals were performed using 1H-1H TOCSY, 1H-13C HSQC spectra, in-house and online databases.

The 1H 1D NMR spectra were directly exported to AMIX 3.8 software (Bruker Biospin GmbH, Karlshure, Germany) and divided into 0.001 ppm-width buckets. In order to remove the effects of possible variations in the water suppression efficiency, the region between 4.67 and 4.92 ppm was discarded. The obtained NMR dataset X-matrix (28 observations×8251 buckets) was then normalised to the total spectrum intensity and then subjected to multivariate statistical analysis using the software SIMCA-P+v.14 (Umetrics, Umeå, Sweden). Initially, the principal component analysis (PCA) of the 1H NMR spectral data was carried out to check the homogeneity of the dataset. Following PCA, a supervised orthogonal partial least squares discriminant analysis (OPLSDA) was then applied to the X-matrix, in which we defined a Y-matrix as the matrix of sample classes, i.e. 0 and 1 for the WT and VIVA groups, respectively. The resulting score and loading plots were used to visualize the discriminant features. A leave-one out internal cross-validation was performed in order to calculate R2Y and Q2 values representing, respectively, the explained variance of the Y matrix and the predictive ability of the model. Model validation was performed by random permutation of the Y matrix with n=999 times and by the use of a CV-ANOVA p value from SIMCA-P+v.14 (analysis of variance in the cross-validated residuals of a Y variable). In addition, VIP (Variable Importance for the Projection) values of metabolites were assigned by using SIMCA-P+v.14 and those variables with VIP larger than 1 are considered relevant for group discrimination.

### LC-MS Metabolomics

Metabolomics profiling of fecal content of VIVA and CEA+Pan-treated WT mice was investigated by the LC-HRMS method (i.e 2 chromatographic columns, in positive ionization mode for the C18 column, and negative for the Zic-p-HILIC column) developed at CEA/SPI (Centre de Saclay, Gif-sur-yvette, France). Mechanical lyse extraction of metabolites was done with the Precellys Soft Tissue homogenizing CK14 BertinPharma and starting with 10 mg of feces in a mixture of H2O 20%/Methanol 80%. All data processing were carried out at CEA/SPI including automatic integration of peaks, selection of relevant variables, annotation with internal databases (mz and RT), and statistical multivariate (PCA-PLS) and univariate (Wilcoxon test) analysis.

### Measure of Urinary Concentrations of Indoxyl Sulfate.

Human urines were collected through the I-BANK IBD cohort generated at the Department of gastroenterology University Hospital of Nancy (Pr. L. Peyrin-Biroulet). Indoxyl sulfate was measured in urine in mice and human using the Indicant Assay kit (Sigma) according to the manufacturer's instructions. The data processing was done by establishing a calibration curve.

### Monitoring of Vnn1 Protein and Pantetheinase Activity.

The anti-mouse Vnn1 ELISA was performed using two specific anti-mouse Vnn1 mAbs (407, rat IgG1, κ; and 24B1, rat IgG2a, κ) as described in Rommelaere et al. [61].

Pantetheinase activity was measured with the pantothenate-7-amino-4-methylcoumarin substrate (Ruan et al, 2010). Tissues were disrupted and lysed with a homogenizer in PBS, 0.1% deoxycholate in the presence of protease inhibitor (Roche). After centrifugation at 10,000 g for 10 min, total protein concentration was measured in the supernatants using the BCA reagent (Pierce Thermo Scientific). Pantetheinase activity was measured by incubating 20-50 μg of total proteins in a final volume of 200 μl phosphate buffer (pH 8) containing 0.01% BSA, 1% DMSO, and 0.0025% Brij 35, 500 μM DTT for 10 min at RT before addition of 20 μM pantothenate-7-amino-4-methylcoumarin. The appearance of AMC was followed during the first 60 min of the reaction by scoring fluorescent signals at 355 nM using a fluorimeter (Tecan) and the slope corresponding to the production rate of the product of the reaction directly reflects the level of enzymatic activity. as described in Rommelaere et al. [61].

### Statistical Analysis.

Results are expressed as means±standard error. Statistical analysis was performed using the GraphPad Prism for Windows software. Statistical significance of the data was compared using the student's t test or 2-way ANOVA. Differences were considered significant at p<0.05*; p<0.01**; p<0.001***

### Results

High VNN1 Expression Correlates with IBD Severity.

We previously shown that VNN1 is highly expressed in colon biopsies from IBD patients [34]. This finding was confirmed by the exploration of Sanofi's Array Land database that includes multiple transcriptomic studies on larger cohorts of IBD patients (data not shown). To study the variations in VNN1 expression at different stages of the disease and under therapy, we prepared a new biobank of colon biopsies obtained from staged CD and UC patients, under various therapies, and controls (data not shown). We then performed RNAseq and bioinformatics analyses from these biopsies. Systematic comparison of all samples using the EGSEA method documented the evolution of the transcriptional signature at different stages of the disease (data not shown). As anticipated, patients with active disease display a dominant cytokine-driven inflammatory signature. A reinforced inflammatory pattern and signs of a strong tissue disorganization accompany failure of anti-TNF biologics. Remarkably, CD patients with clinically resting disease already show enhanced metabolic and repair signatures that reflect mechanisms induced to cope with an infraclinical mucosal stress. In addition to the down-regulation of the inflammatory signature, anti-TNF responders also loose these metabolic and repair signatures, suggesting that the attenuation of inflammation by anti-TNF biologics contributed to tissue recovery (data not shown).

Here we show that VNN1 expression levels increase with disease severity, in both CD and UC (data not shown). Indeed, whereas VNN1 was barely detectable during the quiescent phases of the diseases, its expression augmented during the flares and reached the highest levels in patients resistant to anti-TNFα biologics. We previously showed in mouse that VNN1 is a PPARgamma target gene but also a negative regulator of its expression and activation by pharmacological agonists [33, 34]. Strikingly, in IBD samples, PPARgamma expression level is the reverse mirror of VNN1 expression, and high VNN1 expression level is associated with a dramatic decrease in PPARgamma transcripts (data not shown). Therefore, we anticipated that PPARgamma-dependent transcriptional signatures might be altered in VNNIhigh patients. We used the WGCNA clustering method to group genes with a similar expression profile at different stages of disease evolution (data not shown). In this non-supervised study, we identified a module that contain a geneset displaying a VNN1-like expression pattern (data not shown) and highly enriched in inflammatory and immune markers (data not shown). In contrast, PPARgamma was clustered in a distinct module functionally associated with markers of detoxification and lipid metabolism (data not shown). Consequently, in severe IBD patients with low PPARgamma expression, a subset of bona fide target genes associated with metabolic and repair programs was under expressed (data not shown). This feature was also observed in other colonic pathologies (data not shown). Among them, the reduced expression of the carnitine transporter SLC22A5 and the glucuronidation enzyme UGT1A9 suggested that fatty acid oxidation and detoxification pathways might be impaired, respectively [36, 37]. Similarly, reduction in KLF4 and TSC22D1 expression might reflect a more global impairment of colonocyte differentiation and polarization [38, 39]. In contrast, several other target genes including VNN1 are dissociated from PPARgamma down-regulation and remain highly expressed.

Altogether, colonic VNN1 overexpression is a feature of IBD patients and reflects the degree of tissue inflammation and damage. However, although its contribution to the inflammatory process in gut diseases is related to tissue adaptation to stress, its precise involvement remains unexplained.

### Vnn1 Overexpression by Gut Epithelial Cells Confers Protection to DSS-Induced Colitis.

To mimic the situation observed in IBD patients and explore the contribution of Vnn1 overexpression in gut colitis, we generated the VIVA transgenic mouse (for “VIllin-VAnin-1”) in which the villin promoter specifically drives Vnn1 overexpression in intestinal epithelial cells (data not shown). Quantification of Vnn1 protein levels and pantetheinase activity confirms a high expression only in the gut mucosa, similarly to that observed in the colon of IBD patients (data not shown). Then, VIVA mice were submitted to DSS-induced colitis. Analysis of standard clinical parameters to score the severity of colitis show that DSS-fed VIVA mice displayed reduced weight loss from day 5 to 10 (data not shown) and reduced shortening of the colon (data not shown) compared to DSS-fed control mice. The difference in weight between the two genotypes persisted after DSS withdrawal during the recovery phase (d10-d17). At the microscopic level, DSS-colitis is characterized by a combination of epithelial ulcerations, crypt damage and infiltration of immune cells in the mucosa and submucosa. Histological scoring of tissue damage and inflammation was performed on whole colon sections at day 7 of DSS treatment (data not shown). As expected, colons from DSS-treated control mice showed a higher colitis grade and activity index when compared to VIVA mice (mean values colitis grade: 11.33 vs 9.5 [p=0.0001]; activity index: 18.3 vs 9.5 [p=0.0001]). Inflammation was systematically coupled to ulcerative foci and crypt damage in control mice, whereas in VIVA colons we could observe areas of crypt loss without epithelial ulceration.

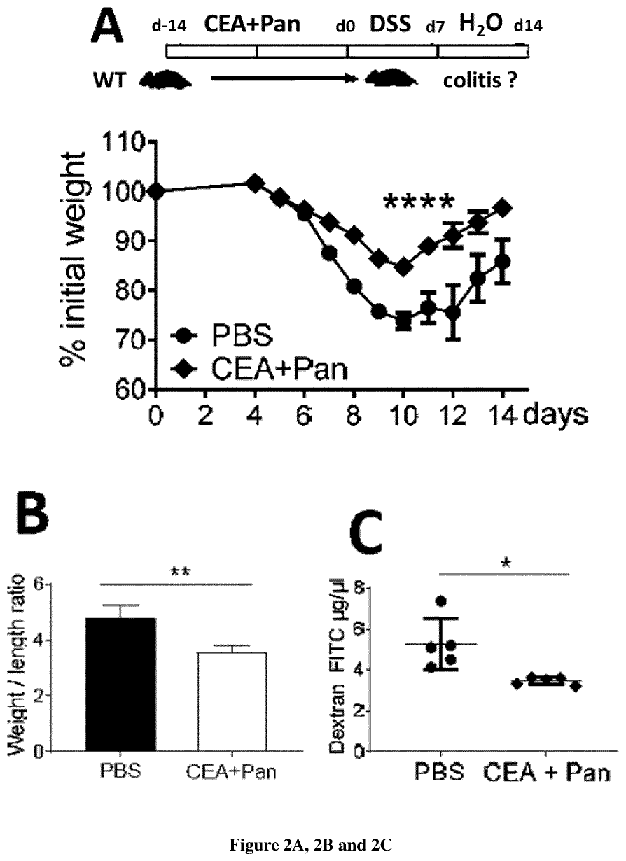

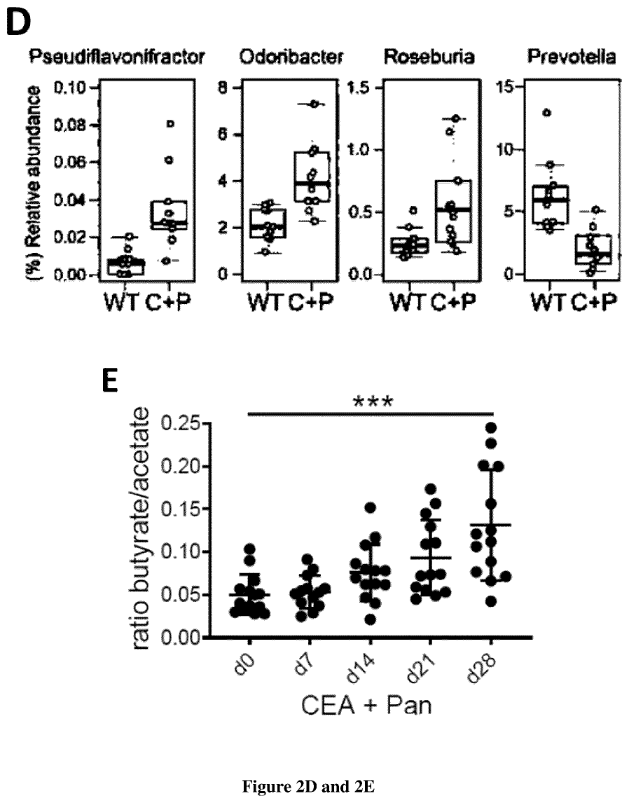

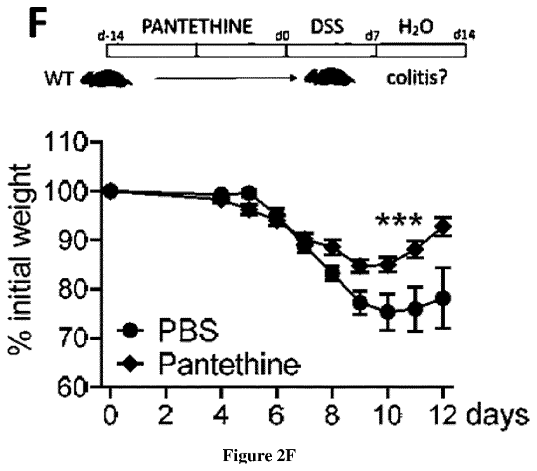

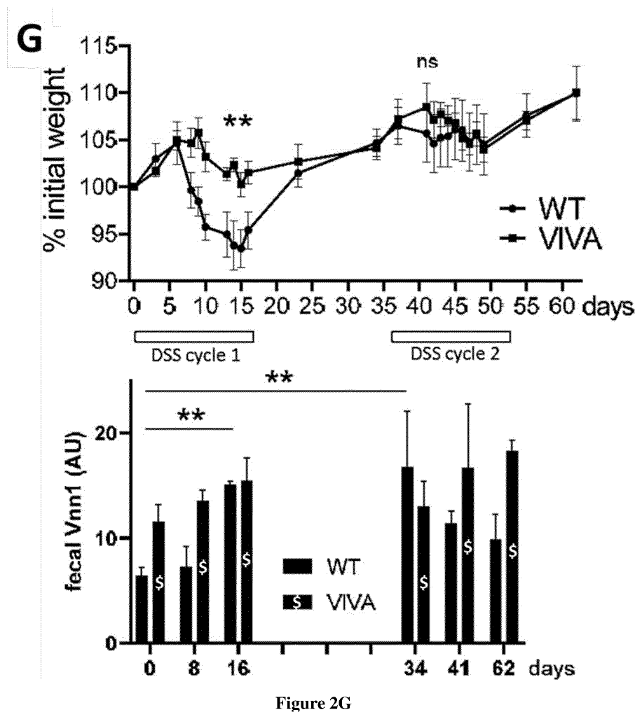

We then scored various markers associated with the integrity of the mucosal barrier in colon. As shown in FIG. 2F-G, the proportion of Ki67+ cells in the crypts under basal conditions is identical in both genotypes. During colitis, the proportion of cycling crypt cells is significantly enhanced in control but not VIVA colons (mean values 72.5% vs 40.6% of proliferative cells/crypt [p=0.0088]) suggesting an exacerbated compensatory epithelial cell proliferation. In contrast, the levels of expression of genes associated with epithelial functions was systematically higher in VIVA versus control colons at day 7 of DSS-colitis (data not shown). This include the epithelial marker villin [40], the anion transporter slc26a3 known to regulate the intracellular pH of colonocytes and whose loss is associated with colitis

susceptibility [41], and IL18 involved in epithelial homeostasis, antimicrobial response and goblet cell function [42].

Next, we quantified neutrophil infiltration by IHC on whole colon using swiss-roll preparations at day 7. The proportion of submucosal and mucosal Ly6G+ infiltrating neutrophils was lower in VIVA compared to controls (mean values 153.9 vs 90.8 cells/field in control vs VIVA mice, respectively [p<0.0001]), in agreement with the reduced degree of tissue damage in these mice (data not shown). Altogether, these results indicate that although inflammation develops in VIVA colon, mucosal integrity is better preserved from DSS-induced damage, requiring less repair processes. The latter depends on the CCL2-mediated recruitment of tissue repair monocytes [43]. Accordingly, we observed reduced levels of MCP1/CCL2 chemokine transcripts and of infiltrating CD64+ lamina propria gut monocytes in VIVA colons compared to controls (data not shown). No difference in other gut immunocyte subpopulations was detectable at that time (not shown).

Thus, increased levels of Vnn1 on intestinal epithelial cells in mice limit the development of DSS-induced epithelial lesions, immune-mediated damage and disease severity.

Improved gut barrier in VIVA mice.

To evaluate the functional impact of Vnn1-induced changes on the mucosal barrier, we scored in vivo intestinal barrier permeability at day 5 of DSS treatment using the orally administered FITC-labeled dextran method. The concentration of serum FITC-dextran was higher in control than VIVA mice (mean values 14.9 μg/ml vs 10.6 μg/ml [p=0.0139]), suggesting that overexpression of Vnn1 on gut epithelial cells restrains the DSS-induced increase of intestinal permeability (data not shown).

The mucus layer is a major regulator of barrier integrity in the colon, and changes in

mucus structure or abundance are markers of IBD [44]. Goblet cells secrete several glycoproteins participating to this physical barrier. Using the PAS-staining method that detects mucosal polysaccharides we observed a higher abundance of PAS+ goblet cells/crypt in VIVA sections compared to WT (mean values 30.9 vs 44.2 in control vs VIVA mice, respectively [p<0.0001]) (data not shown). The enhanced mucus level in VIVA colon lysates was confirmed using PAS-coloured membrane blots (data not shown). We also quantified the expression of Muc2/4, major components of colon mucus, by immunofluorescence on tissue (data not shown) and qRT-PCR on purified colonocytes (data not shown). Both experiments confirmed the increase in Muc levels in VIVA mice.

Finally, we performed an ultrastructural study using electron microscopy to quantify mucus deposition at the apical surface of colonocytes. Mean values of mucus thickness were 183 nm vs 324 nm in control vs VIVA mice, respectively [p=0.0004], data not shown). We then looked at the intracellular stores and release of mucus release by goblet cells at the base of the colonic crypts. Indeed, DSS is known to induce intense secreting activity leading to mucus depletion in goblet cells [45]. On day 2 of DSS exposure, VIVA crypts still harbor heavily loaded goblet cells whereas WT crypt goblet cells are depleted of mucin (data not shown). Consequently, half of the WT but no VIVA crypts were colonized by invading bacteria (data not shown). Furthermore, we detected an increased production of goblet cell-derived antimicrobial peptides (ITLN1, ANG4, RETNLB) in VIVA mice (data not shown). Thus, Vnn1 overexpression is associated with enhanced production of numerous effector proteins associated with barrier integrity and associated functions.

### Improved Colonocyte Fitness and Tolerance to Stress in VIVA Mice.

As recently shown in a tumor model, the Vnn1 pantetheinase contributes to vitamin B5 and CoA regeneration [46]. We show here that CoA levels are significantly elevated in VIVA colon tissue (data not shown). Given the key role of CoA in energetic metabolism and ATP-dependent processes, we probed the global level of protein translation in colon using the puromycin-based SUnSET (Surface SEnsing of Translation) method [47]. Western blot analysis (data not shown) and immunofluorescence staining (data not shown) showed an increased level of puromycin staining in VIVA colons, reflecting enhanced protein synthesis. To estimate mitochondrial activity in situ, we stained purified colonocytes with the MitoSox probe. VIVA colonocytes showed enhanced MitoSox staining indicative of a higher rate of mitochondrial metabolism. Since a shift towards anaerobic glycolysis is often associated with tissue inflammation, we measured lactate production in dissociated colonocytes (data not shown). Interestingly, at basal state, the amount of lactate is lower in VIVA than control colonocytes (data not shown). Upon colitis, a DSS-induced increase in lactate levels was observed in control but not VIVA colons suggesting that the glycolytic “switch” could only be detected in control colons under inflammatory conditions.

We anticipated that this global change in colonocyte fitness should be reflected at the transcriptional level. We focused our analysis on colonocytes at basal state versus early stage of colitis development, i.e. after 4 days of DSS administration and before the development of overt inflammation. We performed a microarray analysis from total RNAs prepared from laser microdissected samples of colon epithelial layer (data not shown). Interestingly, under basal conditions, VIVA colonocytes already displayed a differential epithelial signature compared to control colonocytes (data not shown). It should be noted that the expression of PPARgamma was inversely correlated to that of Vnn1 (data not shown) unlike that of its other target gene SLC22A5 (data not shown). The up-regulated gene signature in VIVA colonocytes is indicative of a trophic epithelial response to growth factors and cytokines (EGF, TGFb, MAPK, Stat3) and control of cell proliferation (Hippo, Id1), in agreement with the increased metabolic rate and epithelial fitness of VIVA colonocytes (data not shown). Of note, we also found an enrichment of the gene set associated with responses to various drugs already shown to modulate Vnn1 expression in mouse [29, 30, 33].

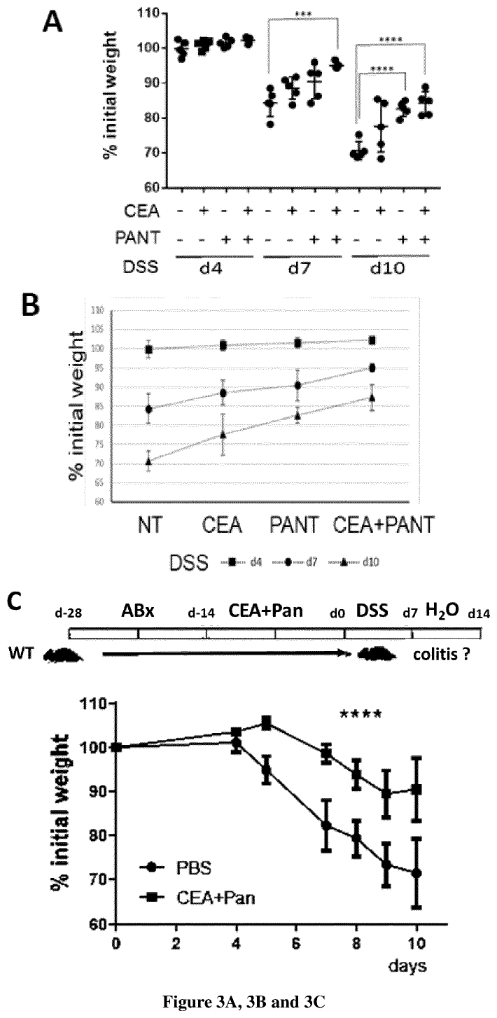

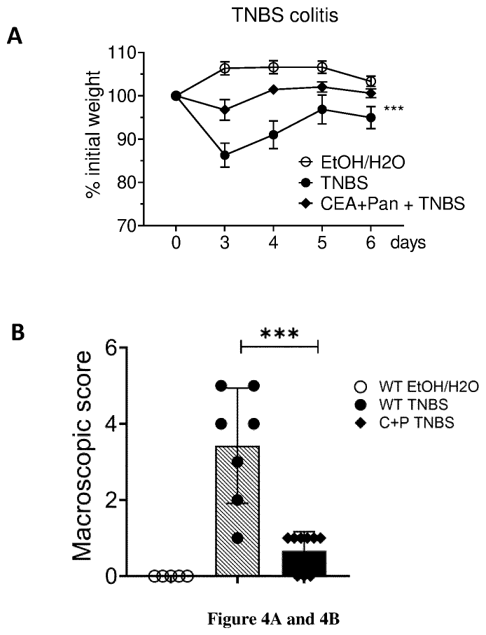

Upon DSS exposure, control colonocytes differentially expressed genes representative of a transcriptomic response to stress (WT_DSS_UP) (data not shown). Interestingly, using the GSEA method this gene set was significantly overrepresented in samples from UC or CD human patients (data not shown). This epithelial signature is therefore present in patients but often masked by the inflammatory response. In contrast, this response was not retrieved from DSS-exposed VIVA colonocytes (WT_DSS_UP against VIVA_CT vs VIVA_DSS) (FIG. 4J-K). The least reactive genes in VIVA colonocytes showed a profile evoking innate immune functions, GPCR signaling and growth and metabolic deregulation (cancer, insulin, calcium, etc.) (data not shown).

Overall, the data argue for a global enhancement of tissue tolerance to stress in VIVA colons linked to the maintenance of a higher metabolic and energetic level.

### Dysbiosis and Enhanced Fecal Butyrate Production in VIVA Mice.

Susceptibility to colitis depends on numerous interactions of gut microbiota with the mucosa [8]. Interestingly, we noticed that upon cohousing of WT and VIVA mice, the susceptibility of WT mice to DSS colitis was reduced to a level comparable to that of VIVA mice, all other parameters such as mouse number per cage, food availability being strictly identical (data not shown). This suggested that the VIVA environment might exert a dominant and transferable phenotype on the susceptibility to colitis of control mice. Therefore, analysis of microbiota from fecal samples was performed on control, VIVA and Vnn1KO mice under homeostatic conditions by sequencing the V3-V4 region of the 16S rRNA gene. While a similar distribution of the main phyla was observed between the different mice groups, microbiota diversity assessed with the Shannon index was significantly reduced in VIVA samples (data not shown) and bacterial composition significantly differed between groups affecting both genera and molecular species (OTUs) (data not shown). More specifically, VIVA mouse microbiota showed significantly increased proportions of the anti-colitis and SCFAs (short-chain fatty acids) producers Barnesiella and Pseudoflavonifractor and conversely, decreased percentages of Eubacterium (data not shown) [48]. This result suggests that the level of Vnn1 at the brush border of colonocytes influences the homeostasis of colonic bacterial communities. Microbiota exchange numerous biological material and metabolites with the mucosa [49]. We speculated that Vnn1 overexpression might in fine impact the composition of fecal metabolome through a modification of the composition and/or metabolic activity of the microbiota. To explore this issue, we used a Nuclear Magnetic Resonance (NMR)-based metabolomic approach for a comparative study between control and VIVA fecal samples under resting conditions. This study showed that Vnn1 levels influenced the respective concentrations of a variety of metabolites which could lead to the segregation of two distinct groups (VIVA vs WT control) by OPLS-DA analysis [p=0.002] (data not shown). More precisely, the fecal metabolome of VIVA mice was enriched in SCFA metabolites (acetate, butyrate, and propionate) compared to that of control samples (data not shown). In particular, analysis of the ratios between butyrate and acetate or propionate showed that butyrate levels were significantly increased in VIVA derived fecal extracts. (data not shown).

Butyrate plays a critical role in the maintenance of the colonic epithelium, and exert multiple actions in cellular processes [50]. It is the most important energetic resource for colonocytes and modulates intracellular signalling [50]. Accordingly, GSEA of VIVA versus WT samples highlighted the upregulation of a geneset associated with the response to sodium butyrate in colonic epithelial cells (data not shown) [51]. To document a butyrate-dependent effect of VIVA fecal extracts on colonocytes, we exposed in vitro Caco2 cells to filtered VIVA or control fecal extracts and scored by qRT-PCR the induction of the CYP1A1 butyrate-responder transcript [52]. As expected, VIVA extracts induced higher CYP1A1 gene expression

than the WT controls (data not shown). Since butyrate should also participate to mitochondrial catabolic activity, we quantified MitROS production using the MitoSox probe, using the complex III inhibitor Antimycin A as a control MitROS inducer. We confirmed that VIVA fecal extracts enhanced MitoSox fluorescence in Caco2 cell cultures compared to WT extracts, reaching a level comparable to that induced by exogenously added butyrate (data not shown). Thus, the protection of the VIVA mice against colitis is linked to dysbiosis and a skewed metabolomic profile in feces leading to enhanced butyrate levels and consequently colonocyte energetic metabolism.

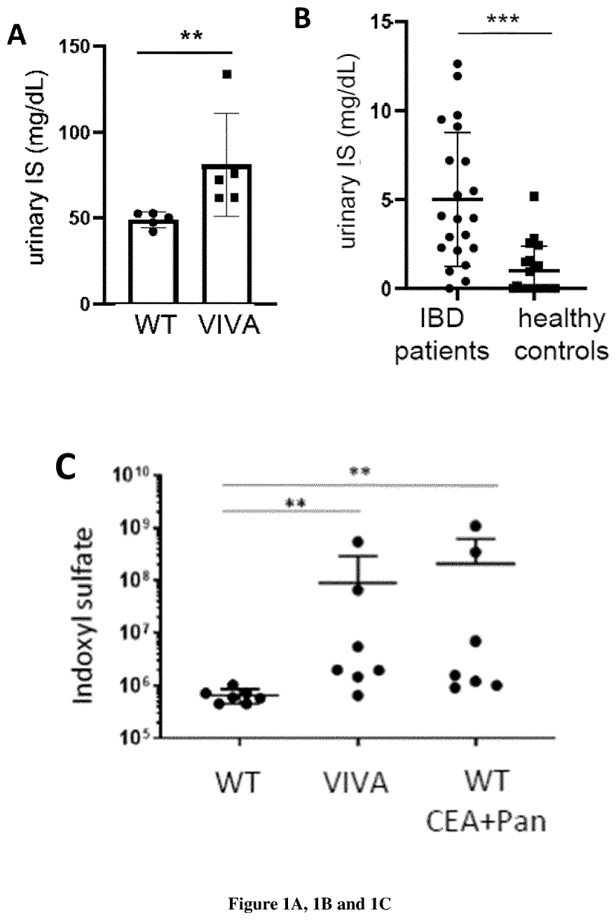

Surprisingly, extended metabolomic analysis of fecal samples using liquid chromatography-mass spectrometry (LC-MS) technology documented high levels of indoxyl sulfate (IS) in the feces of VIVA and CEA+Pan treated WT mice (FIG. 1C). We confirmed the presence of elevated levels of IS in the urine of VIVA mice (FIG. 1A). VNN1 overexpression is a hallmark of IBD and interestingly we found significantly augmented levels of IS in IBD patients compared to controls (mean values: 5 mg/dL vs 1.39 mg/dL, respectively [p<0.0001]),) (FIG. 1B).

### Administration of Pantetheinase Metabolites Recapitulates the Protective Phenotype in WT Mice.

All our analyses rely on the use of VIVA mice that constitutively express Vnn1 at a high level since birth. This situation could profoundly and durably alter mucosal and/or microbial homeostasis in the gut of these mice. To demonstrate that the products of pantetheinase activity can transiently regulate gut homeostasis in normal mice, we chose to develop a preclinical model by administering for a short period these compounds to C57BL/6 mice, expecting that they should pharmacologically mimic the effect of the Vnn1 overexpression. As shown in FIG. 2A-C preconditioning of WT mice by ip injection of CEA (120 mg/kg) and Pan (500 mg/kg) (identified as CEA+Pan therapy) for 15 days prior to DSS administration lead to a milder colitis featuring reduced weight loss and preserved colonic length, similar to that observed in the VIVA mice. Interestingly, this treatment had no effect if animals were treated for shorter periods before or during the DSS protocol (not shown). In addition, whereas CEA alone had a poor influence on DSS-colitis, Pan delayed colitis development and its effect was enhanced by coadministration of CEA (FIG. 3A-B). Accordingly, we detected a reduced intestinal permeability at day 4 of DSS in the treated compared to non-treated mice (data not shown). Moreover, CEA+Pan therapy recapitulated transcriptional changes observed in VIVA mice i.e overexpression of epithelial markers and down-regulation of genes involved in monocyte cells recruitment (data not shown).

We then tested whether CEA+Pan therapy also affected microbiota homeostasis and function. Treated WT mice developed a dysbiosis enriched in SCFAs and butyrate producers (Odoribacter, Pseudoflavonifractor), previously shown to be reduced in IBD patients [48, 53], and conversely, impoverished in bacterial species (Prevotella) associated with susceptibility to colitis (FIG. 2D) [54]. Fecal NMR analysis performed during the time course of CEA+Pan

therapy revealed a progressive increase in butyrate concentrations (FIG. 2E) leading to an increased butyrate to acetate ratio comparable to that measured in VIVA feces. Thus, the pharmacological administration of pantetheinase products recapitulates the mechanisms of protection against colitis conferred by the overexpression of the Vnn1 enzyme in the gut epithelium.

Then, we wondered whether CEA+Pan therapy could recondition the microbiota towards a protective microbial environment. To do so, we depleted mouse microbiota by antibiotherapy prior to CEA+Pan therapy, i.e during the restoration period of the microbiota. As previously reported, WT mice treated with broad-spectrum antibiotics features aggravated DSS-colitis (data not shown) [55]. However, WT mice that have received CEA+Pan therapy after the antibiotherapy, displayed an enhanced protection against colitis (FIG. 3C). This suggested that CEA+Pan therapy allows a facilitated recovery of the gut mucosal barrier post-antibiotherapy, probably by influencing microbiota resilience. The beneficial effect of the combined supply in CEA and Pan was also validated in the TNBS-induced colitis model (FIG. 4A and 4B).

To demonstrate that Vnn1 hydrolytic activity is required for this protective effect, we tested whether administration of the pantetheinase substrate could also mediate the protective effect. We first tested the susceptibility of Vnn1-deficient C57BL/6 mice to DSS colitis and showed that lack of Vnn1 aggravates the severity to colitis (data not shown). Then, we administrated pantethine, a stable dimeric form of the Vnn1 substrate PanSH, to WT and C57BL/6 Vnn1-KO mice. Pantethine enhanced tolerance to DSS-colitis only when a functional Vnn1 was present, i.e in WT but not Vnn1-KO mice (data not shown), confirming that the enzymatic activity of Vnn1 is mandatory for the protection.

Thus, Vnn1 and pantetheinase-associated metabolites contribute to the maintenance of colon homeostasis by modulating microbial ecology, at least partially by controlling butyrate supply and consumption to produce energy in colonocytes. These results demonstrate that the Vnn1/pantheinase pathway can be pharmacologically manipulated for an overall conditioning of the colonic mucosa thereby eliciting a protective effect in colitis.

Vnn1 is Induced by Inflammatory Stress to Cope with Mucosal Injury.

In an acute colitis model, Vnn1 behaves as a cytoprotective molecule. To evaluate its effect in chronic colitis that better recapitulates the situation observed in IBD patients, we used DSS-based models of chronic inflammation by subjecting the mice to 2 cycles of exposure to reduced doses of DSS (0.75%) for longer periods of time (16d) over 48 days. In this model, VIVA mice were again protected during inflammatory episodes (FIG. 2G). Interestingly, in this model of attenuated colonic inflammation, control mice developed virtually no sign of colitis during the 2nd cycle of DSS induction. Since Vnn1 is stress-inducible and can be detected in fecal samples, we monitored by ELISA Vnn1 levels in feces over time. Fecal Vnn1 levels were increased during the 1st inflammatory cycle and were maintained at a level comparable to that observed in VIVA mice during the 2nd cycle. This suggests that the induction of Vnn1 by inflammatory stress is part of a tissue adaptation process intended to allow a better intestinal tolerance to the pathology.

In vitro Study on Mouse Colon Organoids.

We set up an in vitro study on mouse colon organoids. To reproduce the situation in human, the organoids were exposed to TNF over several days before being removed to mimic a targeted anti-TNF immuno-intervention. We then followed the period of mucosal reconstitution post TNF removal by comparing organoids treated or not with pantethine, the substrate of Vnn1 activity.

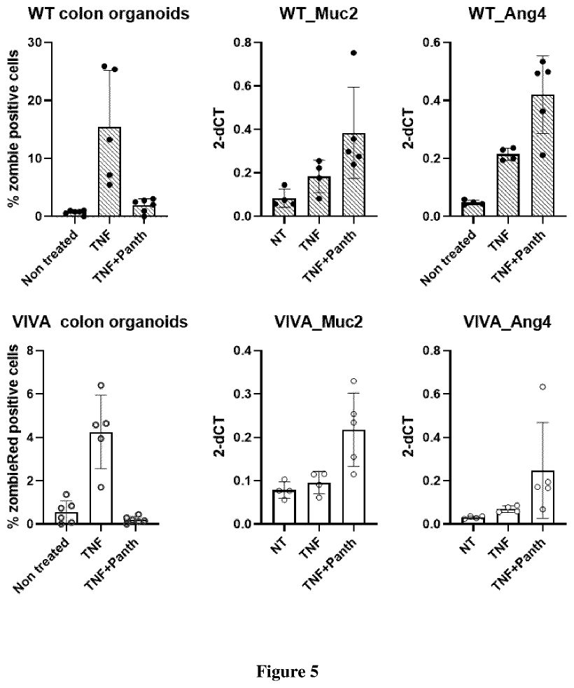

As expected, we observed an increase in cell death following TNF exposure (% zombie red positive cells, FIG. 5). Treatment with pantethine significantly reduced the mortality rate, documenting the protective effect of pantetheinase activity. Furthermore, during the reconstitution phase, we observed an increase in the expression of epithelial genes such as Muc2 and the antimicrobial peptide Ang4, that was further enhanced by addition of pantethine, indicating of a stimulating effect on the epithelial restitution (FIG. 5). TNF exposure of VIVA colon organoids was also correlated with increased cell death (% zombie red positive cells) but less than in WT organoids (FIG. 5). Interestingly, the addition of pantethine almost completely abolished this effect. This strongly suggests that despite the high expression of Vnn1, it is necessary to provide enough substrate to engender a cytoprotective effect. This situation is reminiscent of that encountered in humans. Similarly, addition of pantethine demonstrated a stimulating effect on the Muc2 and Ang4 expression (FIG. 5).

Therefore, combining anti-TNF therapy to pantetheinase derivatives supply may sustain intestinal mucosa recovery.

### DISCUSSION

Our current work clearly established that high VNN1 expression is associated with the degree of inflammation, disease severity and response to therapy in IBD patients. VNN1 has been identified as a biomarker associated with several inflammatory disorders in human [32, 34, 56-58] but its contribution to gut inflammation is still unresolved. In mouse, our former studies performed in Vnn1-deficient BALB/c animals showed that Vnn1 exerted a proinflammatory role in various colitis or CAC models [31, 33, 35]. Mechanistically, Vnn1 was identified as a regulator of GSH levels in the liver and PPARgamma activation in the colon [28, 33]. One limit of these studies was the use of a systemic knockout of the Vnn1 gene, therefore impacting all tissues throughout mouse development. The second limit was linked to the mouse genetic background. Indeed, we later found that control BALB/c mice have lower GSH levels in the liver than C57BL/6 mice at basal stage whereas GSH levels of BALB/c Vnn1 KO mice were identical to that of control C57BL/6 or Vnn1-deficient backcrossed on a C57BL/6 background. Therefore, part of the proinflammatory role of Vnn1 in the BALB/c background could be due to its global impact on systemic GSH homeostasis. Indeed, we show here that Vnn1-deficient C57BL/6 mice are more susceptible to DSS colitis than control mice. This observation is compatible with the fact that under basal conditions, control C57BL/6 mice express very low levels of Vnn1 in the colon, quasi comparable to that of KO mice whereas under DSS stimulation Vnn1 expression is induced and contributes to the cytoprotection of colonocytes, as already observed in the NOD mouse in the context of type 1 diabetes [30]. Therefore, mouse context modulates the effect of variations in Vnn1 levels on the balance between cytoprotection and inflammation.

Since IBD patients display variable levels of VNN1 expression at different stages of the disease, we developed the transgenic VIVA model that overexpresses Vnn1 only in gut epithelial cells and particularly in colon. In this model, Vnn1 exerts a predominant cytoprotective role on colonocytes. We found that at basal stage, Vnn1 overexpression globally enhanced cell activity-and growth-associated transcriptional profiles suggestive of increased trophism. Several aspects of colonocyte activity were boosted in the presence of high Vnn1 levels including the production of mucus and anti-microbial peptides, the control of permeability, and the induction of several epithelial genes associated with cell fitness. The level of mitochondrial activity and protein translation was augmented, and, under inflamed conditions, Vnn1 overexpression prevented the glycolytic shift associated with tissue response to stress. Most changes were observed early after the induction of colitis, ie. before the development of acute inflammation, indicating that cell fitness was maintained in the presence of high Vnn1 levels. This finding is particularly important when dealing with mucus production. Indeed, whereas the crypts of DSS-treated control mice were rapidly depleted of mucus and invaded by bacteria, VIVA crypts remained bacteria-free for longer periods. Altogether, these changes contributed to the maintenance of gut barrier and limited the translocation of bacteria through the mucosa where they trigger inflammation and the development of colitis.

Vnn1 is the predominant mouse tissue pantetheinase that hydrolyses PanSH into CEA and Pan (vitB5), the precursor of CoA synthesis [26]. In a tumor model, we showed that expression of Vnn1 contributes to the maintenance of high Pan and CoA levels in tumor cells [46]. Here, we could demonstrate that the level of Vnn1 on colonocytes parallels that of CoA in gut tissue, comforting our initial observation. CoA plays a major role in many metabolic pathways but is essential for fatty acid oxidation (FAO) and oxidative phosphorylation (OXPHOS) in the mitochondria. Likewise, we could document an increased MitROS production in colonocytes as well as a global enhancement of protein translation, a process requiring high ATP levels. These results indicate that the presence of Vnn1 reinforces the metabolic activity of colonocytes and antagonizes the DSS-induced metabolic rewiring towards glycolysis. This effect could also be due to CEA, previously shown to partially inhibit glycolytic enzymes [46]. The efficacy of FAO and OXPHOS critically depends on fatty acid (FA) substrates, particularly if glycolysis is reduced. Butyrate is the most important source of colonic SCFA and is the result of fermentative metabolism of several bacterial species present in the microbiota [50]. Interestingly, VIVA mice display a dysbiosis of the microbiota enriched in SCFA-producing bacterial species, often underrepresented in IBD patients. This led to an increase in fecal butyrate levels that boosted the transcription of butyrate target genes in cultured colonocyte cell lines. Although one cannot formally exclude the contribution of other changes in the fecal metabolome, our results suggest that Vnn1 induces a potent synergic effect between butyrate production by microbiota and increased CoA-dependent FAO and OXPHOS in the colonocyte. This virtuous energetic loop may explain their improved fitness and functionality.

Interestingly, high Vnn1 expression was also associated with an increased production of IS in feces. Measure of IS urinary concentrations is employed as surrogate of changes in the gut microflora indicating of expansion of bacterial species exploiting tryptophan fermentation. In most IBD patients tested, IS levels were significantly augmented suggesting that IS concentration could be a relevant marker in the follow-up of patients.

Of major interest, we could demonstrated that co-administration of the products of Vnn1 activity (ie. Pan and CEA) to normal mice recapitulates most of the protective phenotypes conferred by the constitutive overexpression of Vnn1 in the VIVA model. Furthermore, CEA+Pan-treated mice display also an enrichment in SCFA-producers and butyrate in the feces, and showed improved microbial resilience after antibiotic therapy in vivo. Although specific studies will be required to explore Vnn1 impact on microbiota, this suggests that Vnn1 products can modulate the microbial ecology at the luminal surface of the gut and might favour growth and/or fermentative activity of particular SCFA-producing bacteria. Remarkably, oral administration of pantethine, the substrate of Vnn1 activity, leads to the same protective result but only if Vnn1 is present in the mouse, confirming the absolute requirement for this enzymatic activity. Further, this argues in favour of an intraluminal effect of Vnn1 where it contributes to epithelial fitness, allowing a better tolerance to inflammatory stress. CEA+Pan-treated mice display an enrichment in SCFA-producers and faecal butyrate, and showed improved microbial resilience after antibiotic therapy in vivo. This demonstrates that Vnn1 products can modulate the microbial ecology at the luminal surface of the gut and favour growth and/or fermentative activity of particular bacteria.

Our results argue that Vnn1 expression by colonocytes promotes microbial adaptation to beneficial SCFA-producing bacterial strains, probably through the delivery of Pan and CEA to the colonic lumen. This is associated with reduced diversity in the faecal microbiota, which is generally considered as detrimental. Under physiological conditions, high colonic expression of Vnn1 is not observed. While the Vnn1-driven enrichment in SCFA-producing strains confers a significant improvement in tolerance to colitis, it might not recapitulate the benefits of a normal microbiota under unchallenged conditions. In favour of this hypothesis, we observed that VIVA mice gain less weight than control mice during their development (not shown).

These results raise an apparent paradox when comparing mouse and human data. Indeed, patients showing the highest levels of VNN1 expression have a more severe disease and often develop a resistance of TNF alpha biologics, probably as a compensatory mechanism. This hypothesis is based on the cytoprotective effect conferred by the pantetheinase activity of Vnn1, as demonstrated in VIVA mice and TNF-treated colonic organoids. Increased pantetheinase activity also impacts the composition of microbial communities and butyrate production, a PPARγ activator. We interpret the human/mouse discrepancy as follows: under physiological conditions, food and microbiota provide an appropriate supply in CEA and Pan. In severe IBD, this metabolic environment becomes progressively impoverished, and the high induction of VNN1 is no longer protective due to the reduced supply of substrate. Santoru et al have reported a Pan deficit in patients with UC and CD, and this observation was replicated in another multi-omics study. In support of this interpretation, we showed that addition of pantethine to VIVA organoids further enhanced cytoprotection and/or repair potential. In a durable inflammatory context, Vnn1-dependent protection becomes insufficient, and this could be due to the significant reduction in PPARγ expression observed in some patients, a phenotype induced by Vnn1 overexpression in mouse. Therefore, combining anti-TNF therapy with pantetheinase derivatives supply may sustain intestinal mucosa recovery.

### REFERENCES

Throughout this application, various references describe the state of the art to which this invention pertains. The disclosures of these references are hereby incorporated by reference into the present disclosure.
1. Kaser, A., S. Zeissig, and R.S. Blumberg, Inflammatory bowel disease. Annu Rev Immunol, 2010. 28: p. 573-621.
2. Kaplan, G.G. and J.W. Windsor, The four epidemiological stages in the global evolution of inflammatory bowel disease. Nat Rev Gastroenterol Hepatol, 2021. 18(1): p. 56-66.
3. Papamichael, K., et al., Infliximab in inflammatory bowel disease. Ther Adv Chronic Dis, 2019. 10: p. 2040622319838443.
4. Billmeier, U., et al., Molecular mechanism of action of anti-tumor necrosis factor antibodies in inflammatory bowel diseases. World J Gastroenterol, 2016. 22(42): p. 9300-9313.
5. Chudy-Onwugaje, K.O., et al., A State-of-the-Art Review of New and Emerging Therapies for the Treatment of IBD. Inflamm Bowel Dis, 2019. 25(5): p. 820-830.
6. Neurath, M.F., Targeting immune cell circuits and trafficking in inflammatory bowel disease. Nat Immunol, 2019. 20(8): p. 970-979.
7. Oka, A. and R.B. Sartor, Microbial-Based and Microbial-Targeted Therapies for Inflammatory Bowel Diseases. Dig Dis Sci, 2020. 65(3): p. 757-788.
8 Plichta, D.R., et al., Therapeutic Opportunities in Inflammatory Bowel Disease: Mechanistic Dissection of Host-Microbiome Relationships. Cell, 2019. 178(5): p. 1041-1056.
9. Battat, R., et al., Histologic Healing Rates of Medical Therapies for Ulcerative Colitis: A Systematic Review and Meta-Analysis of Randomized Controlled Trials. Am J Gastroenterol, 2019. 114(5): p. 733-745.
10. Dal Buono, A., et al., Histological healing: should it be considered as a new outcome for ulcerative colitis? Expert Opin Biol Ther, 2020. 20(4): p. 407-412.
11. Neurath, M.F. and S.P. Travis, Mucosal healing in inflammatory bowel diseases: a systematic review. Gut, 2012. 61(11): p. 1619-35.
12. Iizuka, M. and S. Konno, Wound healing of intestinal epithelial cells. World J Gastroenterol, 2011. 17(17): p. 2161-71.
13. Odenwald, M.A. and J.R. Turner, The intestinal epithelial barrier: a therapeutic target? Nat Rev Gastroenterol Hepatol, 2017. 14(1): p. 9-21.
14. Vancamelbeke, M., et al., Genetic and Transcriptomic Bases of Intestinal Epithelial Barrier Dysfunction in Inflammatory Bowel Disease. Inflamm Bowel Dis, 2017. 23(10): p. 1718-1729.
15. Allaire, J.M., et al., The Intestinal Epithelium: Central Coordinator of Mucosal Immunity. Trends Immunol, 2018. 39(9): p. 677-696.
16. Parikh, K., et al., Colonic epithelial cell diversity in health and inflammatory bowel disease. Nature, 2019. 567(7746): p. 49-55.
17. Smillie, C.S., et al., Intra-and Inter-cellular Rewiring of the Human Colon during Ulcerative Colitis. Cell, 2019. 178(3): p. 714-730 e22.
18. Haberman, Y., et al., Ulcerative colitis mucosal transcriptomes reveal mitochondriopathy and personalized mechanisms underlying disease severity and treatment response. Nat Commun, 2019. 10(1): p. 38.
19. Pisani, L.F., et al., Proteomic insights on the metabolism in inflammatory bowel disease. World J Gastroenterol, 2020. 26(7): p. 696-705.
20. Planell, N., et al., Transcriptional analysis of the intestinal mucosa of patients with ulcerative colitis in remission reveals lasting epithelial cell alterations. Gut, 2013. 62(7): p. 967-76.
21. Litvak, Y., M.X. Byndloss, and A.J. Baumler, Colonocyte metabolism shapes the gut microbiota. Science, 2018. 362(6418).
22. Parada Venegas, D., et al., Short Chain Fatty Acids (SCFAs)-Mediated Gut Epithelial and Immune Regulation and Its Relevance for Inflammatory Bowel Diseases. Front Immunol, 2019. 10: p. 277.
23. Donohoe, D.R., et al., The microbiome and butyrate regulate energy metabolism and autophagy in the mammalian colon. Cell Metab, 2011. 13(5): p. 517-26.
24. Kaiko, G.E., et al., The Colonic Crypt Protects Stem Cells from Microbiota-Derived Metabolites. Cell, 2016. 165(7): p. 1708-1720.
25. Naquet, P., et al., Regulation of coenzyme A levels by degradation: the ‘Ins and Outs’. Prog Lipid Res, 2020. 78: p. 101028.
26. Naquet, P., et al., Role of the Vnn1 pantetheinase in tissue tolerance to stress. Biochem Soc Trans, 2014. 42(4): p. 1094-100.
27. Pitari, G., et al., Pantetheinase activity of membrane-bound Vanin-1: lack of free cysteamine in tissues of Vanin-1 deficient mice. FEBS Lett, 2000. 483(2-3): p. 149-54.
28. Berruyer, C., et al., Vanin-1−/− mice exhibit a glutathione-mediated tissue resistance to oxidative stress. Mol Cell Biol, 2004. 24(16): p. 7214-24.
29. Ferreira, D.W., et al., Enhanced hepatotoxicity by acetaminophen in Vanin-1 knockout mice is associated with deficient proliferative and immune responses. Biochim Biophys Acta, 2016. 1862(4): p. 662-669.
30. Roisin-Bouffay, C., et al., Mouse vanin-1 is cytoprotective for islet beta cells and regulates the development of type 1 diabetes. Diabetologia, 2008. 51(7): p. 1192-201.
31. Martin, F., et al., Vanin-1 (−/−) mice show decreased NSAID-and Schistosoma-induced intestinal inflammation associated with higher glutathione stores. J Clin Invest, 2004. 113(4): p. 591-7.
32. Kavian, N., et al., Imbalance of the Vanin-1 Pathway in Systemic Sclerosis. J Immunol, 2016. 197(8): p. 3326-3335.
33. Berruyer, C., et al., Vanin-1 licenses inflammatory mediator production by gut epithelial cells and controls colitis by antagonizing peroxisome proliferator-activated receptor gamma activity. J Exp Med, 2006. 203(13): p. 2817-27.
34. Gensollen, T., et al., Functional polymorphisms in the regulatory regions of the VNN1 gene are associated with susceptibility to inflammatory bowel diseases. Inflamm Bowel Dis, 2013. 19(11): p. 2315-25.
35. Pouyet, L., et al., Epithelial vanin-1 controls inflammation-driven carcinogenesis in the colitis-associated colon cancer model. Inflamm Bowel Dis, 2010. 16(1): p. 96-104.
36. Juraszek, B. and K.A. Nalecz, SLC22A5 (OCTN2) Carnitine Transporter-Indispensable for Cell Metabolism, a Jekyll and Hyde of Human Cancer. Molecules, 2019. 25(1).
37. Mizuma, T., Intestinal glucuronidation metabolism may have a greater impact on oral bioavailability than hepatic glucuronidation metabolism in humans: a study with raloxifene, substrate for UGT1A1, 1A8, 1A9, and 1A10. Int J Pharm, 2009. 378(1-2): p. 140-1.
38. Gupta, R.A., et al., Peroxisome proliferator-activated receptor gamma and transforming growth factor-beta pathways inhibit intestinal epithelial cell growth by regulating levels of TSC-22. J Biol Chem, 2003. 278(9): p. 7431-8.
39. Yu, T., et al., Kruppel-like factor 4 regulates intestinal epithelial cell morphology and polarity. PLOS One, 2012. 7(2): p. e32492.
40. Wang, Y., et al., A novel role for villin in intestinal epithelial cell survival and homeostasis. J Biol Chem, 2008. 283(14): p. 9454-64.
41. Schweinfest, C.W., et al., slc26a3 (dra)-deficient mice display chloride-losing diarrhea, enhanced colonic proliferation, and distinct up-regulation of ion transporters in the colon. J Biol Chem, 2006. 281(49): p. 37962-71.
42. Nowarski, R., et al., Epithelial IL-18 Equilibrium Controls Barrier Function in Colitis. Cell, 2015. 163(6): p. 1444-56.
43. Farro, G., et al., CCR2-dependent monocyte-derived macrophages resolve inflammation and restore gut motility in postoperative ileus. Gut, 2017. 66(12): p. 2098-2109.
44. Sheng, Y.H., et al., Mucins in inflammatory bowel diseases and colorectal cancer. J Gastroenterol Hepatol, 2012. 27(1): p. 28-38.
45. Dharmani, P., P. Leung, and K. Chadee, Tumor necrosis factor-alpha and Muc2 mucin play major roles in disease onset and progression in dextran sodium sulphate-induced colitis. PLOS One, 2011. 6(9): p. e25058.
46. Giessner, C., et al., Vnn1 pantetheinase limits the Warburg effect and sarcoma growth by rescuing mitochondrial activity. Life Sci Alliance, 2018. 1(4): p. e201800073.
47. Schmidt, E.K., et al., SUnSET, a nonradioactive method to monitor protein synthesis. Nat Methods, 2009. 6(4): p. 275-7.
48. Polansky, O., et al., Important Metabolic Pathways and Biological Processes Expressed by Chicken Cecal Microbiota. Appl Environ Microbiol, 2015. 82(5): p. 1569-76.
49. Blacher, E., et al., Microbiome-Modulated Metabolites at the Interface of Host Immunity. J Immunol, 2017. 198(2): p. 572-580.
50. Liu, H., et al., Butyrate: A Double-Edged Sword for Health? Adv Nutr, 2018. 9(1): p. 21-29.
51. Tabuchi, Y., et al., Genetic networks responsive to sodium butyrate in colonic epithelial cells. FEBS Lett, 2006. 580(13): p. 3035-41.
52. Jin, U.H., et al., Short Chain Fatty Acids Enhance Aryl Hydrocarbon (Ah) Responsiveness in Mouse Colonocytes and Caco-2 Human Colon Cancer Cells. Sci Rep, 2017. 7(1): p. 10163.
53. Morgan, X.C., et al., Dysfunction of the intestinal microbiome in inflammatory bowel disease and treatment. Genome Biol, 2012. 13(9): p. R79.
54. Scher, J.U., et al., Expansion of intestinal Prevotella copri correlates with enhanced susceptibility to arthritis. Elife, 2013. 2: p. e01202.
55. Perse, M. and A. Cerar, Dextran sodium sulphate colitis mouse model: traps and tricks. J Biomed Biotechnol, 2012. 2012: p. 718617.
56. Kang, M., et al., VNN1, a potential biomarker for pancreatic cancer-associated new-onset diabetes, aggravates paraneoplastic islet dysfunction by increasing oxidative stress. Cancer Lett, 2016. 373(2): p. 241-50.
57. Washino, S., et al., A Novel Biomarker for Acute Kidney Injury, Vanin-1, for Obstructive Nephropathy: A Prospective Cohort Pilot Study. Int J Mol Sci, 2019. 20(4).
58. Zhu, X. and R.S. Cooper, Admixture mapping provides evidence of association of the VNN1 gene with hypertension. PLoS One, 2007. 2(11): p. e1244.
59. Santoru, M.L., et al., Cross sectional evaluation of the gut-microbiome metabolome axis in an Italian cohort of IBD patients. Sci Rep, 2017. 7(1): p. 9523.
60. Albillos A, de Gottardi A, Rescigno M. The gut-liver axis in liver disease: Pathophysiological basis for therapy. J Hepatol. 2020 March; 72(3): 558-577.
61. Mønsted MØ, Falck ND, Pedersen K, Buschard K, Holm LJ, Haupt-Jorgensen M. Intestinal permeability in type 1 diabetes: An updated comprehensive overview. J Autoimmun. 2021 August; 122:102674.
62. Xia L., et al. The Role for Gut Permeability in the Pathogenesis of Type 1 Diabetes—A Solid or Leaky Concept? Pediatr Diabetes. 2015 November; 16(7): 485-492.
63. Allam-Ndoul B, Castonguay-Paradis S, Veilleux A. Gut Microbiota and Intestinal Trans-Epithelial Permeability. Int J Mol Sci. 2020 Sep. 3;21(17): 6402.
64. Zia K, Ashraf S, Jabeen A, Saeed M, Nur-E-Alam M, Ahmed S, Al-Rehaily AJ, Ul-Haq Z. Identification of potential TNF-α inhibitors: from in silico to in vitro studies. Sci Rep. 2020 Dec. 1;10(1): 20974.
65. Gareb B, Otten AT, Frijlink HW, Dijkstra G, Kosterink JGW. Review: Local Tumor Necrosis Factor-a Inhibition in Inflammatory Bowel Disease. Pharmaceutics. 2020 Jun. 11;12(6): 539.

1. A method for repairing an intestinal mucosal barrier in a subject in need thereof comprising administering to said subject an effective amount of at least one agent of vanin-1 pantetheinase (Vnn1) metabolism selected from the group consisting of i) pantethine, ii) pantothenate and/or a pantothenate analog, iii) cysteamine, iv) pantothenate and cysteamine, and v) a pantothenate analog and cysteamine.

2. The method according to claim 1, wherein the subject is a human afflicted with or susceptible to be afflicted with an intestinal barrier defects.

3. The method according to claim 3, wherein the subject is a human afflicted with or susceptible to be afflicted with inflammatory bowel disease.

4. A method for treating disease associated with intestinal barrier defects in a subject in need thereof comprising administering to the subject a therapeutically effective amount of at least one agent of Vnn1 metabolism selected from the group consisting of i) pantethine, ii) pantothenic acid and/or a pathotenic acid analog, iii) cysteamine, iv) pantothenic acid and cysteamine, and v) a pantothenic acid analog and cysteamine.

5. The method according to claim 4, wherein the disease associated with intestinal barrier defects is inflammatory bowel disease (IBD), irritable bowel syndrome (IBS), chronic liver disease, type 1 diabetes or obesity.

6. The method according to claim 5, wherein the inflammatory bowel disease (IBD) is Crohn's disease (CD) or ulcerative colitis (UC).

7. The method according to claims 1, wherein the at least one agent of Vnn1 metabolism is administered in combination with a therapeutic compound used to treat disease associated with intestinal barrier defects.

8. The method according to claim 7, wherein the therapeutic compound used to treat disease associated with intestinal barrier defects is an anti-TNF alpha compound.

9. A pharmaceutical composition comprising at least one agent of Vnn1 metabolism selected from the group consisting of i) pantethine, ii) pantothenate and/or a pantothenate analog, iii) cysteamine, iv) pantothenate and cysteamine, and v) a pantothenate analog and cysteamine for use for repairing intestinal mucosal barrier in subject in need thereof.

10. The method according to claim 1, wherein the at least one agent of Vnn1 metabolism is pantothenate and cysteamine.

11. The method according to claim 1, wherein the at least one agent of Vnn1 metabolism is a pantothenate analog and cysteamine.

12. The method according to claim 11, wherein the pantothenate analog is dexpanthenol.

13. A pharmaceutical composition comprising at least one agent of Vnn1 metabolism compound(s) selected from the group consisting of i) pantethine, ii) pantothenic acid and/or a pathotenic acid analog, iii) cysteamine, iv) pantothenic acid and cysteamine, and v) a pantothenic acid analog and cysteamine for use for treating disease associated with intestinal barrier defects in a subject in need thereof.

14. An in vitro method for diagnosing inflammatory bowel disease in a subject and treating the subject, comprising i) determining in a sample obtained from a subject, the expression level of indoxyl sulfate; and ii) administering to a subject identified as having an expression level of indoxyl sulfate that is higher than a corresponding reference value a pharmaceutical composition comprising at least one agent of Vnn1 metabolism.

15. The in vitro method according to claim 14, wherein the sample is a feces sample or a urinary sample.
